(19) United States
(12) Patent Application Publication
(10) Pub. No.: US 2011/0150805 A1
(43) Pub. Date: Jun. 23, 2011

(54) MAKEUP COMPOSITION

(75) Inventors: Guillaume KERGOSIEN, Chaville (FR); Nathalie JAGER LEZER, Verrieres-Le-Buisson (FR); Emmanuelle PORTOIS, Choisy Le Roi (FR)

(73) Assignee: L'OREAL, Paris (FR)

(21) Appl. No.: 12/969,621

(22) Filed: Dec. 16, 2010

Related U.S. Application Data
(60) Provisional application No. 61/300,460, filed on Feb. 2, 2010.
(60) Provisional application No. 61/300,461, filed on Feb. 2, 2010.
(60) Provisional application No. 61/300,462, filed on Feb. 2, 2010.

(30) Foreign Application Priority Data
Dec. 18, 2009 (FR) 09 59173
Dec. 18, 2009 (FR) 09 59177
Dec. 18, 2009 (FR) 09 59178

(51) Int. Cl. A61K 8/92; A61Q 1/14; A61Q 1/10

(52) U.S. Cl. 424/70.7; 424/78.03

(57) ABSTRACT

Composition for making up keratin fibres, such as eyelashes or eyebrows, having improved makeup-removing properties, containing in a continuous aqueous phase:
an aqueous dispersion of a particular polyurethane present in an amount of solids greater than or equal to 5% by weight relative to the total weight of said composition, and
said composition comprising an emulsifying system comprising less than 2% by weight of triethanolamine.

### REFERENCE TO PRIOR APPLICATIONS

This application claims priority to U.S. provisional application Ser. Nos. 61/300,460, 61/300,461, and 61/300,462, all filed Feb. 2, 2010, and to French patent applications 09 59173, 09 59177, and 09 59178, all filed Dec. 18, 2009, all incorporated herein by reference.

### FIELD OF THE INVENTION

The present invention relates to a composition for making up keratin materials, suitable for forming makeup films on keratin fibres, such as the eyelashes or eyebrows, and that can easily be removed.

The film of makeup may be formed from a composition that is in the form of a mascara, or a product for the eyebrows. More preferably, the invention relates to the removal and/or cleansing of a mascara. The term “mascara” is understood to mean a composition intended to be applied to the eyelashes: it may be an eyelash makeup composition, an eyelash makeup base (also known as basecoat), a composition to be applied to a mascara, also known as topcoat, or else a composition for the cosmetic treatment of the eyelashes. The mascara is more particularly intended for the eyelashes of humans, but also for false eyelashes.

### BACKGROUND OF THE INVENTION

Eyelash makeup compositions or mascaras may be constituted of at least one wax or of a mixture of waxes dispersed in an aqueous or organic solvent liquid phase. In general they have a pasty texture and are packaged in a container. This container defines a reservoir equipped with a wiper and sealed by an applicator. This applicator may especially be in the form of a brush or a comb configured in order to withdraw the product contained in the reservoir. The excess product can then be removed from such an applicator when it passes through the wiper and such an applicator can then be brought into contact with keratin fibres to be made up, such as the eyelashes or eyebrows.

However, the film of makeup obtained after the application of these compositions is not generally sufficiently resistant to water, for example when bathing or taking showers, to tears or to sweat or else to sebum. The mascara then has a tendency to run—appearance of rings under the eyes—or to disintegrate over time: grains are deposited and unattractive traces appear around the eyes.

In order to solve the aforementioned problem, polyurethane dispersions have recently been incorporated into the cosmetic products offering several advantages compared to conventional technologies such as acrylics and acrylamide copolymers, polyvinyl pyrrolidone and PVP/VA copolymers. These advantages include compatibility with water, water resistance and excellent ability to form films. However, such dispersions may suffer from a lack of adherence to the coated surface which results in disintegration of the composition. This creates a significant aesthetic problem for the consumers. Such examples of polyurethane dispersions are in particular provided in applications EP 1 970 391 and EP 2 105 126.

U.S. Pat. No. 6,106,813 also discloses polyester polyurethanes which are suitable in cosmetic applications. It discloses a novel family of polyester polyurethanes which has not only good film-forming properties, but which also confers great rigidity and excellent resistance to removal by water and detergents.

This type of composition thus, in general, makes it possible to obtain a film that has good non-transfer properties, good properties of resistance over time, in particular to water and to rubbing, and that forms a comfortable deposit on the skin, the lips, the eyelashes or the nails. Thus, this new formulation pathway makes it possible to improve the performances of products for making up, caring for or treating keratin materials.

On the other hand, the films formed by such dispersions are more difficult to remove than conventional products.

One objective of the present invention therefore lies in the development of a makeup composition which, while retaining good hold on the eyelashes or eyebrows, or even improving this hold, furthermore has good makeup-removing ability.

Consequently, there remains a need to provide compositions that form makeup films that have good hold while being easily removable.

### DETAILED DESCRIPTION OF THE PREFERRED EMBODIMENTS

One subject of the present invention is a composition for making up keratin fibres, such as eyelashes or eyebrows, having improved makeup-removing properties, comprising in a continuous aqueous phase:
an aqueous dispersion of polyurethane, the dispersed polyurethane comprising the reaction products of:

A) a prepolymer according to the formula:

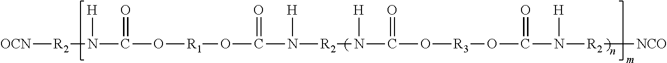

where:

R₁ represents a hydrocarbon-based radical derived from a polyester polyol, and in particular from a polyester diol,

R₂ represents a hydrocarbon-based radical derived from an aliphatic or cycloaliphatic polyisocyanate,

R₃ represents a hydrocarbon-based radical derived from a diol, optionally of low molecular weight, optionally substituted by ionic groups,

n is equal to 0 to 5, and

m is >1;

B) at least one chain extender according to the formula:

H₂N—R₄—NH₂

where

R₄ represents an alkylene or alkylene oxide radical that is not substituted by ionic or potentially ionic groups; and

C) at least one chain extender according to the formula:

H₂N—R₅—NH₂

where

R₅ represents an alkylene radical substituted by ionic or potentially ionic groups;
said polyurethane being present in an amount of solids greater than or equal to 5% by weight relative to the total weight of said composition,

said composition comprising an emulsifying system comprising less than 1% by weight of triethanolamine, and comprising at least one dyestuff chosen from pulverulent materials.

The present invention also relates to a method for the removal from and/or cleansing of keratin fibres, such as the eyelashes or eyebrows, of a makeup composition as defined previously applied to the eyelashes, said method comprising a step of applying a makeup-removing and/or cleansing composition to the film of makeup.

Said method may optionally comprise a step of exerting tension on the film of makeup from the base of the keratin fibre to the free end of said fibre with a view to at least partially removing said film. This step may be carried out manually and in particular using an optionally laminated, woven or non-woven support, such as a Demakeup® disc. This support may be anhydrous. As a variant, this support may be pre-impregnated with the makeup-removing and/or cleansing composition. This makeup-removing and/or cleansing composition may be water or a water-soluble solvent.

The makeup-removing and/or cleansing compositions may comprise water and/or one or more water-soluble solvent(s), for example present in an amount ranging from 50 to 100% by weight relative to the total weight of the composition. These compositions may also comprise an oily phase.

Such compositions may be in the form of lotions, optionally two-phase lotions, milks, creams or else gels.

The inventors have discovered that these particular makeup films may easily be removed by a conventional pulling movement enabling makeup removal in the form of a sheath, without substantial disintegration of the makeup films. Such makeup removal thus makes it possible to avoid dispersing the makeup composition during its removal from the keratin fibres.

The term “sheaths” is understood to mean the formation of sleeves having a length greater than or equal to 1 mm, better still of at least 2 mm.

In order to evaluate the makeup-removing and/or cleansing result of the composition according to the present application, an in vitro test is carried out according to the following protocol:
the composition is applied to three samples of straight Caucasian hair of 30 knots (60 eyelashes having a length of 1 cm), fringe length of 2 cm, by making 3×10 passes at 2 minute intervals with uptake of product between each series of 10 passes.
Each sample is then dried at room temperature for a drying time of one hour.
The 3 made-up samples are immersed in a vessel containing water at 20° C. for a given time (1 hour, 24 hours or a week). The 3 samples are then wiped back and forth 5 times on a square cloth of the Wypall L40 type from Kimberly Clark.
The 3 samples are then deposited on a respective cotton support of Demakeup® disc type, then each cotton pad is folded in 2 around their respective sample.
A slight pressure is applied to each cotton pad and said cotton pads are pulled relative to the samples.
The deposit on each cotton pad is then observed visually.

The present invention also relates to an assembly or kit for removing makeup from keratin fibres, such as eyelashes or eyebrows, comprising:
at least one makeup composition described above, and
at least one composition for the removal and/or cleansing of this makeup composition.

The polyurethane dispersions of the present invention are particularly suitable for use in products for making up keratin materials and in particular keratin fibres, in particular eyelashes or eyebrows.

A subject of the present invention is, according to a second subject optionally independent of the emulsifying system used, a composition for making up and/or caring for keratin fibres, such as the eyelashes or eyebrows, comprising in a continuous aqueous phase:

A) a prepolymer according to the formula:

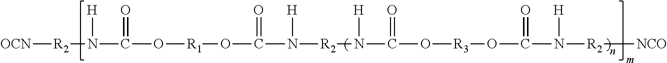

where:

R₁ represents a divalent hydrocarbon-based radical or a dihydroxyl-functional compound, advantageously a radical derived from a polyester polyol, and in particular from a polyester diol,

R₂ represents a hydrocarbon-based radical derived from an aliphatic or cycloaliphatic polyisocyanate,

R₃ represents a hydrocarbon-based radical derived from a diol, optionally of low molecular weight, optionally substituted by ionic groups,

n is equal to 0 to 5, and

m is >1;

B) at least one chain extender according to the formula:

H₂N—R₄—NH₂

where

R₄ represents an alkylene or alkylene oxide radical that is not substituted by ionic or potentially ionic groups; and

C) at least one chain extender according to the formula:

H₂N—R₅—NH₂

where

R₅ represents an alkylene radical substituted by ionic or potentially ionic groups; and

at least one hydrophilic thickener chosen from the copolymers derived from the polymerization:

(i) of at least one monomer of formula (1) below:

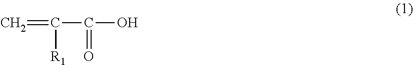

in which, R₁ denotes H or CH₃ or C₂H₅, that is to say acrylic acid, methacrylic acid or ethacrylic acid monomers, and

(ii) of at least one monomer of (C₁₀-C₃₀)alkyl ester of unsaturated carboxylic acid type corresponding to the monomer of formula (2) below:

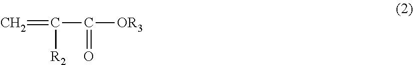

in which, R₂ denotes H or CH₃ or C₂H₅ and preferably H or CH₃, R₃ denoting a C₁₀-C₃₀, and preferably C₁₂-C₂₂, alkyl radical.

Unexpectedly, the inventors have discovered that the use of particular polyurethanes formulated in a continuous aqueous phase in the presence of a hydrophilic thickener as defined previously makes it possible to obtain a composition that has excellent resistance to water and/or to sebum, and where appropriate very good resistance to rubbing.

Advantageously, the (C₁₀-C₃₀)alkyl esters of unsaturated carboxylic acids are preferably chosen from lauryl acrylate, stearyl acrylate, decyl acrylate, isodecyl acrylate, dodecyl acrylate and the corresponding methacrylates, such as lauryl methacrylate, stearyl methacrylate, decyl methacrylate, isodecyl methacrylate and dodecyl methacrylate, and mixtures thereof.

According to one preferred embodiment, these thickening polymers are crosslinked.

According to one particular embodiment, said hydrophilic thickener is chosen from polymers resulting from the polymerization of a mixture of monomers comprising:

(i) essentially acrylic acid,

(ii) an ester of formula (2) described above in which R₂ denotes H or CH₃, R₃ denoting an alkyl radical having from 12 to 22 carbon atoms, and

(iii) a crosslinking agent, such as a copolymerizable polyethylenic unsaturated monomer, for instance diallyl phthalate, allyl (meth)acrylate, divinylbenzene, (poly)ethylene glycol dimethacrylate and methylenebisacrylamide.

Said composition advantageously comprises:

(i) from 95 to 60% by weight of acrylic acid,

(ii) from 4 to 40% by weight of C₁₀-C₃₀ alkyl acrylate, and

(iii) from 0 to 6% by weight of crosslinking polymerizable monomer.

Said composition advantageously comprises:

(i) from 98 to 96% by weight of acrylic acid,

(ii) from 1 to 4% by weight of C₁₀-C₃₀ alkyl acrylate, and

(iii) from 0.1 to 0.6% by weight of crosslinking polymerizable monomer.

Various tests for evaluating this resistance with respect to the prior art have been carried out in order to demonstrate the advantageous effects provided by the proposed technical solution. The results of the tests carried out in relation to this subject of the invention will be presented later on in this description.

Another subject of the present invention is, according to a third subject optionally independent of the emulsifying system used, and optionally independent of the presence or absence of hydrophilic thickener, a composition for making up and/or caring for keratin fibres comprising:
an aqueous polyurethane dispersion, the dispersed polyurethane comprising the reaction products of:

A) a prepolymer according to the formula:

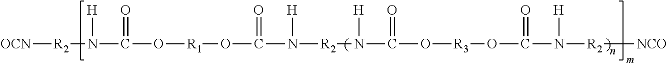

where:

R₁ represents a divalent hydrocarbon-based radical or a dihydroxyl-functional compound, advantageously a radical derived from a polyester diol,

R₂ represents a hydrocarbon-based radical derived from an aliphatic or cycloaliphatic polyisocyanate,

R₃ represents a hydrocarbon-based radical derived from a diol, optionally of low molecular weight, optionally substituted by ionic groups,

n is equal to 0 to 5, and

m is >1;

B) at least one chain extender according to the formula:

H₂N—R₄—NH₂

where

R₄ represents an alkylene or alkylene oxide radical that is not substituted by ionic or potentially ionic groups; and

C) at least one chain extender according to the formula:

H₂N—R₅—NH₂

where

R₅ represents an alkylene radical substituted by ionic or potentially ionic groups; and

at least one lipophilic plasticizer present in an amount by weight greater than or equal to 2% relative to the total weight of the composition.

Unexpectedly, the inventors have discovered that the use of particular polyurethanes formulated in a continuous aqueous phase in the presence of a plasticizer present in an amount by weight strictly greater than 2% relative to the total weight of the composition makes it possible to obtain a composition that has very good resistance to water and/or to sebum, and where appropriate very good resistance to rubbing.

Advantageously, this plasticizer is chosen from:
the (poly)esters derived from the reaction(s) of at least one carboxylic acid with at least one (poly)ol,
glycol ethers,
N-ethyl-o,p-toluenesulphonamide,
carbonates,
ketones,

and mixtures thereof.

According to one preferred embodiment, said plasticizer is chosen from citric acid esters, such as triethyl citrate, tributyl citrate, triethyl acetylcitrate, tributyl acetylcitrate, 2-triethylhexyl acetylcitrate, and mixtures thereof.

Here too, various tests for evaluating this resistance with respect to the prior art have been carried out in order to demonstrate the advantageous effects provided by the proposed technical solution, which will be divulged later on in this description.

Unless otherwise mentioned, the description which follows is adapted to the three independent subjects set out above.

Polyurethanes

The polyurethane may be present in an amount of solids greater than or equal to 5% by weight relative to the total weight of the composition, better still 6.5% by weight or even 8% by weight or more. The polyurethane may, for example, be present in an amount of solids ranging from 5% to 30% by weight relative to the total weight of said composition, better still ranging from 6 to 25%, better still from 7 to 15%.

The compositions according to the invention comprise aqueous dispersions of particles of particular polyurethanes. Such compositions may be used in cosmetic, makeup or care applications for eyelashes or eyebrows.

The polyurethane is a reaction product of:

A) a prepolymer according to the formula:

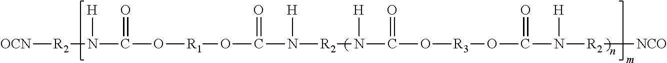

where:

R₁ represents a hydrocarbon-based radical derived from a polyester polyol, and in particular from a polyester diol,

R₂ represents a hydrocarbon-based radical derived from an aliphatic or cycloaliphatic polyisocyanate,

R₃ represents a hydrocarbon-based radical derived from a diol, optionally of low molecular weight, optionally substituted by ionic groups,

n is equal to 0 to 5, and

m is >1;

B) at least one chain extender according to the formula:

H₂N—R₄—NH₂

where

R₄ represents an alkylene or alkylene oxide radical that is not substituted by ionic or potentially ionic groups; and

C) at least one chain extender according to the formula:

H₂N—R₅—NH₂

where

R₅ represents an alkylene radical substituted by ionic or potentially ionic groups.

Prepolymer A)

The compositions according to the invention comprise aqueous dispersions of particular polyurethanes. Such compositions may be used in cosmetic, makeup or care applications for eyelashes or eyebrows.

Radical R₁

Suitable compounds for providing the polyhydroxyl radical, preferably dihydroxyl radical, R₁, are polyester polyols, preferably polyester diols, and mixtures thereof. These compounds are thus advantageously divalent, preferably having two hydroxyl groups.

Such compounds may have number-average molecular weights of around 700 to around 16 000, and preferably of around 750 to around 5000.

The polyester diol(s) may generally be prepared from:
aliphatic, cycloaliphatic or aromatic dicarboxylic or polycarboxylic acids, or anhydrides thereof; and
dihydric alcohols such as diols chosen from aliphatic, alicyclic or aromatic diols.

The aliphatic dicarboxylic or polycarboxylic acids may be chosen from: succinic, fumaric, glutaric, 2,2-dimethylglutaric, adipic, itaconic, pimelic, suberic, azelaic, sebacic, maleic, malonic, 2,2-dimethylmalonic, nonanedicarboxylic, decanedicarboxylic, dodecanedioic, 1,3-cyclohexanedicarboxylic, 1,4-cyclohexane-dicarboxylic, 2,5-norboranedicarboxylic, diglycolic, thiodipropionic, 2,5-naphthalenedicarboxylic, 2,6-naphthalenedicarboxylic, phthalic, terephthalic, isophthalic, oxanic, o-phthalic, tetrahydrophthalic, hexahydrophthalic or trimellitic acid.

The acid anhydrides may, in particular, be chosen from o-phthalic, trimellitic or succinic acid anhydride or a mixture thereof.

Preferably, the dicarboxylic acid is adipic acid.

The dihydric alcohols may be chosen from ethanediol, ethylene glycol, diethylene glycol, triethylene glycol, trimethylene glycol, tetraethylene glycol, 1,2-propanediol, dipropylene glycol, tripropylene glycol, tetrapropylene glycol, 1,3-propanediol, 1,4-butanediol, 1,3-butanediol, 2,3-butanediol, 1,5-pentanediol, 1,6-hexanediol, 2,2-dimethyl-1,3-propanediol, 1,4-dihydroxycyclohexane, 1,4-dimethylolcyclohexane, cyclohexanedimethanol, 1,8-octanediol, 1,10-decanediol, 1,12-dodecanediol, neopentyl glycol or mixtures thereof. The cycloaliphatic and/or aromatic dihydroxyl compounds are, of course, also suitable as the dihydric alcohol(s) for the preparation of the polyester polyol(s).

The polyester diols may also be chosen from homopolymers or copolymers of lactones, which are preferably obtained by addition reactions of lactones or lactone mixtures, such as butyrolactone, ε-caprolactone and/or methyl-ε-caprolactone with the appropriate polyfunctional, preferably difunctional, starter molecules such as, for example, the dihydric alcohols mentioned above. The corresponding polymers of ε-caprolactone are preferred.

The polyester polyol, preferably polyester diol, radical R₁, may advantageously be obtained by polycondensation of dicarboxylic acids, such as adipic acid, with polyols, especially diols, such as hexanediol, neopentyl glycol and mixtures thereof.

Radical R₂

Suitable polyisocyanates for providing the hydrocarbon-based radical R₂ include organic diisocyanates having a molecular weight of around 112 to 1000, and preferably of around 140 to 400.

Preferred diisocyanates are those represented by the general formula R₂(NCO)₂ indicated above, in which R₂ represents a divalent aliphatic hydrocarbon group comprising 4 to 18 carbon atoms, a divalent cycloaliphatic hydrocarbon group comprising 5 to 15 carbon atoms, a divalent araliphatic hydrocarbon group comprising 7 to 15 carbon atoms or a divalent aromatic hydrocarbon group comprising 6-15 carbon atoms. Examples of the organic diisocyanates which are suitable include tetramethylene diisocyanate, 1,6-hexamethylene diisocyanate, dodecamethylene diisocyanate, cyclohexane-1,3-diisocyanate and cyclohexane-1,4-diisocyanate, 1-isocyanato-3-isocyanatomethyl-3,5,5-trimethylcyclohexane (isophorone diisocyanate or IPDI), bis(4-isocyanatocyclohexyl)-methane, 1,3-bis(isocyanatomethyl)cyclohexane and 1,4-bis(isocyanatomethyl)cyclohexane and bis(4-isocyanato-3-methylcyclohexyl)methane. Mixtures of diisocyanates can, of course, be used. Preferred diisocyanates are aliphatic and cycloaliphatic diisocyanates. Particularly preferred are 1,6-hexamethylene diisocyanate, isophorone diisocyanate and dicyclohexylmethane diisocyanate and mixtures thereof.

Radical R₃

The use of diols, especially low molecular weight diols, R₃, may allow a stiffening of the polymer chain, and is optional. The expression “low molecular weight diols” means diols having a molecular weight of around 62 to 700, preferably 62 to 200. They may contain aliphatic, alicyclic or aromatic groups. Preferred compounds contain only aliphatic groups. The diols used preferably have up to 20 carbon atoms and may be chosen from ethylene glycol, diethylene glycol, propane-1,2-diol, propane-1,3-diol, butane-1,4-diol, 1,3-butylene glycol, neopentyl glycol, butylethylpropanediol, cyclohexanediol, 1,4-cyclohexanedimethanol, hexane-1,6-diol, bisphenol A (2,2-bis(4-hydroxyphenyl)propane), hydrogenated bisphenol A (2,2-bis(4-hydroxycyclohexyl)-propane), and mixtures thereof. Preferably, R₃ is derived from neopentyl glycol.

Optionally, the low molecular weight diols may contain ionic or potentially ionic groups. Suitable low molecular weight diols containing ionic or potentially ionic groups are those disclosed in U.S. Pat. No. 3,412,054. Preferred compounds include dimethylol-butanoic acid (DMBA), dimethylolpropionic acid (DMBA) and carboxyl-containing caprolactone polyester diol. If low molecular weight diols containing ionic or potentially ionic groups are used, they are preferably used in an amount such that <0.30 meq of COOH is present per gram of polyurethane in the polyurethane dispersion. Preferably, the low molecular weight diols containing ionic or potentially ionic groups are not used.

The chain of the prepolymer is extended using two classes of chain extenders, B) and C).

B) Chain Extenders

Compounds B) of the first class of chain extender having the formula:

H₂N—R₄—NH₂

where R₄ represents an alkylene or alkylene oxide radical not substituted with ionic or potentially ionic groups.

Thus, the chain extender may be chosen from:
Alkylenediamines such as hydrazine, ethylene-diamine, propylenediamine, 1,4-butylenediamine and piperazine.
Alkylene oxide diamines such as dipropylamine diethylene glycol (DPA-DEG available from Tomah Products, Milton, Wis.), 2-methyl-1,5-pentanediamine (Dytec A from DuPont), hexanediamine, isophorone-diamine, and 4,4-methylenedi(cyclohexylamine), and the DPA-series of ether amines available from Tomah Products, Milton, Wis., including dipropylamine propylene glycol, dipropylamine dipropylene glycol, dipropylamine tripropylene glycol, dipropylamine poly(propylene glycol), dipropylamine ethylene glycol, dipropylamine poly(ethylene glycol), dipropylamine 1,3-propanediol, dipropylamine 2-methyl-1,3-propanediol, dipropylamine 1,4-butanediol, dipropylamine 1,3-butanediol, dipropylamine 1,6-hexanediol and dipropylamine cyclohexane-1,4-dimethanol, and mixtures thereof.

Preferably, the chain extender B) is chosen from ethylenediamine, diethanolamine and mixtures thereof.

C) Chain Extenders

The second class of chain extenders are compounds C) having the formula:

H₂N—R₅—NH₂

where R₅ represents an alkylene radical substituted with ionic or potentially ionic groups. These compounds have an ionic or potentially ionic group and two isocyanate-reactive groups. The ionic group or the potentially ionic group may be chosen from the group constituted by ternary or quaternary ammonium groups, groups convertible into such a group, a carboxyl group, a carboxylate group, a sulphonic acid group and a sulphonate group. Said at least partial conversion of the groups convertible into salt groups of the type mentioned may take place before or during the mixing with water. Specific compounds include diaminosulphonates, such as for example the sodium salt of N-(2-aminoethyl)-2-aminoethanesulphonic acid (AAS) or the sodium salt of N-(2-aminoethyl)-2-aminopropionic acid.

Preferably, R₅ represents an alkylene radical substituted with sulphonic acid or sulphonate groups.

Preferably, this compound is the sodium salt of N-(2-aminoethyl)-2-aminoethanesulphonic acid (AAS).

Chain Terminators

The polyurethane according to the invention may also comprise compounds which are situated in each case at the ends of the chains and terminate said chains.

These chain terminators may be derived from compounds having the formula:

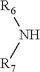

in which R₆ represents a hydrogen atom or an alkylene radical optionally having a hydroxyl end and R₇ represents an alkylene radical optionally having a hydroxyl end. Suitable compounds include compounds such as monoamines, particularly secondary monoamines, or monoalcohols. Examples include: methylamine, ethylamine, propylamine, butylamine, octylamine, laurylamine, stearylamine, isononyloxypropylamine, dimethylamine, diethylamine, dipropylamine, dibutylamine, N-methylaminopropylamine, diethyl-(methyl)aminopropylamine, morpholine, piperidine, diethanolamine and suitable substituted derivatives thereof, amide amines of primary diamines and monocarboxylic acids, monoketimes of primary diamines, primary/tertiary amines such as N,N-dimethylamino-propylamine and the like. Chain terminating alcohols may be chosen from C₁-C₁₀ alcohols, such as methanol, butanol, hexanol, 2-ethylhexyl alcohol, isodecyl alcohol, and mixtures thereof. Amino alcohols such as aminomethylpropanol (AMP) are also suitable.

In one embodiment of the invention, diethylene glycol is used to obtain the polyurethane, either as the low molecular weight diol, or as part of the non-ionic chain extender through the use of dipropylamine diethylene glycol. If the diethylene glycol is used as the low molecular weight diol, then preferably the DPA-DEG is not used as the non-ionic chain extender. Likewise, if the DPA-DEG is used as the non-ionic chain extender, then diethylene glycol is preferably not used as the low molecular weight diol.

Preparation Processes

The compositions according to the invention comprise an aqueous polyurethane dispersion suitable for use in makeup or care products for keratin fibres such as the eyelashes or eyebrows, and are capable of being obtained by a preparation process comprising the following steps:

A) preparation of an aqueous polyurethane dispersion via:

1) formation of an isocyanate-functional prepolymer by reacting:

1a) a polyester polyol, and especially a poly-ester diol;

1b) an aliphatic or cycloaliphatic polyisocyanate, and

1c) a low molecular weight diol, optionally substituted with ionic groups;

2) chain extension of the prepolymer via:

2a) at least one chain extender according to the formula:

H₂N—R₄—NH₂

where R₄ represents an alkylene or alkylene oxide radical not substituted with ionic or potentially ionic groups, and

2b) at least one chain extender according to the formula:

H₂N—R₅—NH₂

where R₅ represents an alkylene radical substituted with ionic or potentially ionic groups, in the presence of an organic solvent in order to form a polyurethane;

3) dispersion of the polyurethane in water; and

4) removal of organic solvent, which makes it possible to obtain an aqueous polyurethane dispersion; and

mixing of the polyurethane dispersion with water or ethanol.

More particularly, a process for producing a polyurethane dispersion suitable for use in makeup products may comprise the following steps: a) reacting, in a first step, at least one polyester polyol compound and a low molecular weight diol that is optionally substituted with an ionic group (dihydroxyl compounds) with diisocyanate to form the prepolymer A), then b) dissolving, in a second step, the prepolymer in an organic solvent and c) reacting, in a third step, the isocyanate-containing prepolymer solution with the two classes of chain extenders and optionally, the chain terminator, d) forming, in a fourth step, the dispersion by addition of water, and e) removing, in a fifth step, the organic solvent.

The free sulphonic acid groups incorporated are neutralized between the third and fourth step. Suitable neutralizing agents included are the primary, secondary or tertiary amines. Of these the trialkyl-substituted tertiary amines are preferred. Examples of these amines are trimethylamine, triethylamine, triisopropylamine, tributylamine, N,N-dimethylcyclohexylamine, N,N-dimethylstearylamine, N,N-dimethylaniline, N-methyl-morpholine, N-ethylmorpholine, N-methylpiperazine, N-methylpyrrolidine, N-methylpiperidine, N,N-dimethyl-ethanolamine, N,N-diethylethanolamine, triethanolamine, N-methyldiethanolamine, dimethylaminopropanol, 2-methoxyethyldimethylamine, N-hydroxyethylpiperazine, 2-(2-dimethylaminoethoxy)ethanol and 5-diethylamino-2-pentanone. The preferred tertiary amines are those which do not contain active hydrogen(s) as determined by the Zerewitinoff test given that the hydrogen can react with the isocyanate groups of the prepolymers which can cause gelation, the formation of insoluble particles or chain termination.

The polyurethane dispersions can be produced by what is known as the acetone process. In the acetone process, the synthesis of the aqueous preparations of polyurethane on which the dispersions according to the invention are based is performed in a multistage process.

In a first stage, a prepolymer containing isocyanate groups is synthesized from the polyester polyol compound, the diisocyanate and the low molecular weight diol. The amounts of the individual components are calculated in such a way that the isocyanate content of the prepolymers is between 1.4 and 5.0 wt %, preferably between 2.0 and 4.5 wt %, and particularly preferably between 2.6 and 4.0 wt %. The low molecular weight diol is present in an amount from 0 to 80 eq % based on the amount of NCO equivalents, preferably from 0 to 10 eq %.

The prepolymer obtained has the structure:

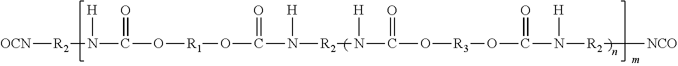

where:

R₁ represents a polyester polyol and in particular a polyester diol,

R₂ represents a hydrocarbon radical or an aliphatic or cycloaliphatic polyisocyanate,

R₃ represents a radical of a low molecular weight diol, optionally substituted with ionic groups,

n is <5, and

m is >1.

Preferably, n is from 1 to 3, and m is from 1 to 5.

In a second stage, the prepolymer produced in stage 1 is dissolved in an organic, at least partially water-miscible, solvent containing no isocyanate-reactive groups. The preferred solvent is acetone. Other solvents, such as, for example, 2-butanone, tetrahydrofuran or dioxane or mixtures of these solvents can also be used, however. The quantities of solvent to be used must be calculated in such a way that a solids content of 25 to 60 wt %, preferably 30 to 50 wt %, particularly preferably 35 to 45 wt %, is obtained.

In a third stage, the isocyanate-containing prepolymer solution is reacted with mixtures of amino-functional chain extenders and, optionally, chain terminators, to form the high molecular weight polyurethane. Sufficient amounts of the chain extenders and chain terminator are used such that the calculated number-average molecular weight (Mₙ) of the polyurethane obtained is between 10 000 and 100 000 daltons, preferably between 10 000 and 50 000 daltons. The non-ionic chain extender is present in an amount from 15 to 90 eq %, preferably 35.0 to 55 eq %, based on the residual amount of NCO equivalents present in the prepolymer. The ionic chain extender is present in an amount from 10 to 50 eq %, preferably from 25 to 35 eq %, based on the residual amount of NCO equivalents present in the prepolymer. The chain terminator is present in an amount from 0 to 35 eq %, preferably from 20 to 30 eq %, based on the residual amount of NCO equivalents present in the prepolymer.

In a fourth stage, the high molecular weight polyurethane is dispersed in the form of a fine-particle dispersion by addition of water to the solution or solution to the water.

In a fifth stage, the organic solvent is partially or wholly removed by distillation, optionally under reduced pressure. The amount of water in stage four is calculated in such a way that the aqueous polyurethane dispersions according to the invention display a solids content of 20 to 60 wt %, preferably 28 to 42 wt %.

Aqueous Phase

The composition according to the invention may comprise an aqueous phase comprising water and/or at least one water-soluble solvent. This aqueous phase is continuous.

The expression “continuous aqueous phase composition” is understood to mean that the composition has a conductivity, measured at 25° C., of greater than or equal to 23 μS/cm (microSiemens/cm), the conductivity being measured, for example, using an MPC227 conductivity meter from Mettler Toledo and an Inlab730 conductivity measurement cell. The measurement cell is immersed in the composition, so as to remove the air bubbles liable to be formed between the two electrodes of the cell. The conductivity is read as soon as the value of the conductivity meter has stabilized. An average is taken over at least 3 successive measurements.

The expression “water-soluble solvent” denotes, in the present invention, a compound that is liquid at ambient temperature and water-miscible (miscibility in water greater than 50% by weight at 25° C. and atmospheric pressure).

The water-soluble solvents that can be used in the compositions according to the invention may also be volatile.

Among the water-soluble solvents which may be used in the compositions according to the invention, mention may especially be made of lower monoalcohols having 1 to 5 carbon atoms such as ethanol and isopropanol, glycols having 2 to 8 carbon atoms such as ethylene glycol, propylene glycol, 1,3-butylene glycol and dipropylene glycol, C₃ and C₄ ketones and C₂-C₄ aldehydes.

The aqueous phase (water and optionally the water-miscible solvent) may be present in the composition in a content ranging from 1% to 95% by weight, relative to the total weight of the composition, preferably ranging from 5% to 80% by weight, and preferentially ranging from 10% to 60% by weight.

The aqueous phase according to the invention may also comprise at least one hydrophilic film-forming polymer and/or at least one hydrophilic thickener and/or at least one surfactant, such as those listed previously. However, the aqueous phase content indicated previously does not include the contents of each of the aforementioned compounds.

Plasticizer

The composition according to the invention may comprise a plasticizer that promotes the formation of a film with the film-forming polymer, in particular with the polyurethane used in the present invention. The plasticizer aims, in particular, to make said polyurethane more flexible, by reducing its glass transition temperature (Tg).

In particular, the plasticizer may advantageously be chosen from lipophilic plasticizers.

The expression “lipophilic plasticizer” denotes, in the present invention, a compound that is liquid at ambient temperature (25° C.), that preferably has a viscosity of less than 100 cPs, preferably of less than 50 cPs, and that has a solubility in water at 25° C. of less than 10%, preferably of less than 5%.

The plasticizer is in particular chosen from the (poly)esters derived from the reaction(s) of at least one carboxylic acid with at least one (poly)ol, glycol ethers, N-ethyl-o,p-toluenesulphonamide, carbonates, ketones, and mixtures thereof.

The term “(poly)ol” is understood to mean a polyol or monoalcohol compound.

The term “polyol” is understood to mean an organic compound comprising at least 2 hydroxyl groups, particularly between 2 and 6 hydroxyl groups, said hydroxyl groups being borne by i) a C₁-C₆ alkyl group optionally interrupted by from 1 to 6 heteroatoms chosen from N, S, O; ii) a C₅-C₂₀ aryl group; iii) a heteroaryl group comprising between 5 and 20 chain members with 1 to 3 heteroatom(s) chosen from N, S, O; iv) a heterocycloalkyl group; and v) a cycloalkyl group. In particular, as a poly(ol), mention may for example be made of ethylene glycol or glycerol.

The term “monoalcohol” is understood to mean an organic compound comprising a single hydroxyl group, said hydroxyl group being borne by i) a C₁-C₆ alkyl group optionally interrupted by from 1 to 6 heteroatoms chosen from N, S, O; ii) a C₅-C₂₀ aryl group; iii) a heteroaryl group comprising between 5 and 20 chain members with 1 to 3 heteroatom(s) chosen from N, S, O; iv) a heterocycloalkyl group; and v) a cycloalkyl group.

The plasticizer may thus be chosen more particularly from:

a) The esters derived from the reaction, and in particular the monocondensation or polycondensation, of one or more carboxylic or orthophosphoric acids with one or more (poly)ol(s), such as a diol. Such esters may be (mono/poly)esters, and preferably polyesters, such as diesters.

Such esters may generally be derived from the reaction of one or more (mono/poly)carboxylic acid(s) of formula R₁₁(COOH)ₙ with one or more (poly)ol(s) of formula R₁₂(OH)ₘ where:
R₁₁ and R₁₂, which are identical or different, represent a saturated or unsaturated, optionally non-aromatic, linear, branched or cyclic C₃-C₁₅ cycloalkyl hydrocarbon-based chain preferably comprising from 3 to 15 carbon atoms, optionally comprising one or more heteroatoms such as N, O, S such as 0 to 6 heteroatoms;
n ranging from 1 to 6; and
m ranging from 1 to 6.

Preferably, R₁₁ is a C₂-C₅ alkyl radical such as ethyl, propyl, butyl, isobutyl and R₁₂ is a branched or saturated linear hydrocarbon-based chain comprising from 5 to 10 carbon atoms.

As examples of esters, mention may in particular be made of:
esters derived from the reaction of R(COOH) where R represents a branched C₃ to C₁₀ alkyl chain such as isobutyl or tert-butyl, with HOC(R)(R′)—C(OH)—CH(R″)—R′″ where R represents a hydrogen atom or a linear or branched alkyl chain such as methyl and R′, R″ and R′″ representing a linear or branched alkyl chain such as methyl. Mention may especially be made of the esters derived from the monocondensation or from the dicondensation of tert-butylic acid and 2,2,4-trimethylpentane-1,3-diol. In particular, mention may be made of a monoester resulting from the reaction of isobutylic acid and octanediol such as 2,2,4-trimethylpentane-1,3-diol, such as TEXANOL Ester Alcohol sold by Eastman Chemical;
phosphoric acid esters derived from the condensation between one or more (poly)ol(s) and one or more orthophosphoric acid(s) of formula (R—Z)ₙ—P═O(O—R′)ₘ with R, R′ representing a linear or branched C₁-C₂₀ alkyl group, n being equal to 1, 2 or 3 and m being equal to 0, 1 or 2, with m+n being equal to 3 and Z being a heteroatom such as oxygen or a sigma bond. By way of example of such esters, mention may be made, inter alia, of tricresyl phosphate, tributyl phosphate, triphenyl phosphate and tributoxyethyl phosphate;
fatty acid esters derived from the (mono/poly)condensation between one or more (poly)ol(s) and one or more fatty acids of formula R(COOH)ₙ with R being a C₃ to C₂₂ saturated linear alkyl chain and n being equal to 1 or 2. By way of example of such esters, mention may be made, inter alia, of adipic acid esters, such as diisobutyl adipate, diethyl adipate or stearic acid esters, such as ethyl stearate, or else palmitic acid esters, such as 2-ethylhexyl palmitate;
citric acid esters or citrates, such as triethylcitrate, tributylcitrate, triethyl acetylcitrate, tributyl acetylcitrate, 2-triethylhexyl acetylcitrate;
phthalic acid esters or phthalates, such as diethyl phthalate, dibutyl phthalate, dioctyl phthalate, dipentyl phthalate, dimethoxyethyl phthalate, butyl phthalate and 2-ethylhexyl phthalate;
tartaric acid esters or tartrates, especially dibutyl tartrate;
sebacic acid esters or sebacates, such as dimethyl sebacates, dibutyl sebacate;
propyleneglycol diacetate and glycerol triacetate;
benzylbenzoate;
butyl acetylricinoleate, glyceryl acetylricinoleate; and
butylglycolate.

b) Glycol Ethers

As examples of glycol ethers, mention may be made of diethylene glycol ethyl ether, diethylene glycol methyl ether, diethylene glycol butyl ether, diethylene glycol hexyl ether, ethylene glycol ethyl ether, ethylene glycol butyl ether, ethylene glycol hexyl ether or else propylene glycol phenyl ether, dipropylene glycol butyl ether, tripropylene glycol butyl ether, propylene glycol methyl ether, dipropylene glycol ethyl ether, tripropylene glycol methyl ether, diethylene glycol methyl ether and propylene glycol butyl ether,

c) Use may also be made, as a plasticizer, of N-ethyl-o,p-toluenesulphonamide and in particular ethyl tosylamide as sold under the reference RESIMPOL 8 by Pan-Americana.

d) Carbonates;

e) Ketones, especially camphor; and mixtures thereof.

The plasticizer(s) may be present in an amount ranging from 1 to 15% by weight relative to the total weight of the composition, preferably from 2 to 10%, and better still from 3 to 8% by weight. Generally, this plasticizer may be present in an amount by weight greater than or equal to, better still strictly greater than, 2% relative to the total weight of the composition.

Surfactants

The composition according to the invention may contain an emulsifying system comprising one or more surfactants present, in particular, in a content ranging from 0.1% to 20% by weight relative to the total weight of the composition, or even 0.5% to 15% by weight, preferably ranging from 1% to 10% by weight.

An emulsifying surfactant suitably chosen in order to obtain an oil-in-water emulsion is generally used. In particular, an emulsifying surfactant having, at 25° C., an HLB balance (hydrophilic-lipophilic balance), within the meaning of Griffin, of greater than or equal to 8 may be used.

The HLB value according to Griffin is defined in J. Soc. Cosm. Chem. 1954 (volume 5), pages 249-256. These surfactants can be selected from nonionic, anionic, cationic or amphoteric surfactants, and mixtures thereof. Reference may be made to the document “Encyclopedia of Chemical Technology, Kirk-Othmer”, volume 22, p. 333-432, 3rd edition, 1979, Wiley, for the definition of the properties and emulsifying functions of surfactants, in particular p. 347-377 of this reference, for anionic, amphoteric and nonionic surfactants.

The surfactants preferably used in the composition according to the invention are selected from:

a) nonionic surfactants with an HLB of greater than or equal to 8 at 25° C., used alone or as a mixture. Mention may in particular be made of:
oxyethylenated and/or oxypropylenated glycerol ethers which may comprise from 1 to 150 oxyethylene and/or oxypropylene units;
oxyalkylenated, in particular oxyethylenated and/or oxypropylenated alcohols, which may comprise from 1 to 150 oxyethylene and/or oxypropylene units, preferably from 20 to 100 oxyethylene units, in particular fatty alcohols, especially ethoxylated C₈-C₂₄ and preferably C₁₂-C₁₈ fatty alcohols, such as the ethoxylated stearyl alcohol comprising 20 oxyethylene units (CTFA name “Steareth-20”), such as BRIJ 78 sold by Uniqema, the ethoxylated cetearyl alcohol comprising 30 oxyethylene units (CTFA name “Ceteareth-30”) and the mixture of C₁₂-C₁₅ fatty alcohols comprising 7 oxyethylene units (CTFA name “C₁₂₋₁₅ Pareth-7”), such as that sold under the name NEODOL 25-7® by Shell Chemicals;
esters of a fatty acid, in particular a C₈-C₂₄ and preferably C₁₆-C₂₂ fatty acid, and of polyethylene glycol (or PEG) (which may comprise from 1 to 150 oxyethylene units), such as PEG-50 stearate and PEG-40 monostearate, sold under the name MYRJ 52P® by Uniqema;
esters of a fatty acid, in particular a C₈-C₂₄ and preferably C₁₆-C₂₂ fatty acid, and of oxyethylenated and/or oxypropylenated glycerol ethers (which may comprise from 1 to 150 oxyethylene and/or oxypropylene units), such as the polyoxyethylenated glyceryl monostearate comprising 200 oxyethylene units sold under the name Simulsol 220 TM® by SEPPIC; polyoxyethylenated glyceryl stearate comprising 30 oxyethylene units, such as the product TAGAT S® sold by Goldschmidt, polyoxyethylenated glyceryl oleate comprising 30 oxyethylene units, such as the product TAGAT O® sold by Goldschmidt, polyoxyethylenated glyceryl cocoate comprising 30 oxyethylene units, such as the product VARIONIC LI 13® sold by Sherex, polyoxyethylenated glyceryl isostearate comprising 30 oxyethylene units, such as the product TAGAT L® sold by Goldschmidt, and polyoxyethylenated glyceryl laurate comprising 30 oxyethylene units, such as the product TAGAT I® from Goldschmidt;
esters of a fatty acid, in particular a C₈-C₂₄ and preferably C₁₆-C₂₂ fatty acid, and of oxyethylenated and/or oxypropylenated sorbitol ethers (which may comprise from 1 to 150 oxyethylene and/or oxypropylene units), such as the polysorbate 60 sold under the name TWEEN 60® by Uniqema;
dimethicone copolyol, such as that sold under the name Q2-5220® by Dow Corning;
dimethicone copolyol benzoate, such as that sold under the name FINSOLV SLB 101® and 201® by Fintex;
copolymers of propylene oxide and of ethylene oxide, also known as EO/PO polycondensates,
and mixtures thereof.

The EO/PO polycondensates are more particularly copolymers consisting of polyethylene glycol and polypropylene glycol blocks, such as, for example, polyethylene glycol/polypropylene glycol/polyethylene glycol triblock polycondensates. These triblock polycondensates have, for example, the following chemical structure:

H—(O—CH₂—CH₂)ₐ—(O—CH(CH₃)—CH₂)_{b}—(O—CH₂—CH₂)ₐ—OH,

in which formula a ranges from 2 to 120 and b ranges from 1 to 100.

The EO/PO polycondensates preferably have a weight-average molecular weight ranging from 1000 to 15 000 and better still ranging from 2000 to 13 000. Advantageously, said EO/PO polycondensates have a cloud point, at 10 g/l in distilled water, of greater than or equal to 20° C., preferably of greater than or equal to 60° C. The cloud point is measured according to the ISO 1065 standard. Mention may be made, as EO/PO polycondensate which can be used according to the invention, of the polyethylene glycol/polypropylene glycol/polyethylene glycol triblock polycondensates sold under the SYNPERONIC® names, such as SYNPERONIC PE/L44® and SYNPERONIC PE/F127®, by ICI.

b) nonionic surfactants with an HLB of less than 8 at 25° C., optionally in combination with one or more nonionic surfactants with an HLB of greater than 8 at 25° C., such as mentioned above, such as:
esters and ethers of saccharides, such as sucrose stearate, sucrose cocoate, sorbitan stearate and mixtures thereof, for example ARLATONE 2121®, sold by ICI, or SPAN 65V, from Uniqema;
esters of fatty acids, in particular C₈-C₂₄ and preferably C₁₆-C₂₂ fatty acids, and of a polyol, in particular of glycerol or of sorbitol, such as glyceryl stearate, sold for example under the name TEGIN M® by Goldschmidt, glyceryl laurate, such as the product sold under the name IMWITOR 312® by Hüls, polyglyceryl-2 stearate, sorbitan tristearate and glyceryl ricinoleate;
oxyalkylenated, in particular oxyethylenated and/or oxypropylenated alcohols, which may comprise from 1 to 15 oxyethylene and/or oxypropylene units, in particular ethoxylated C₈-C₂₄ and preferably C₁₂-C₁₈ fatty alcohols, such as the ethoxylated stearyl alcohol comprising 2 oxyethylene units (CTFA name “Steareth-2”), such as BRIJ 72 sold by Uniqema;
the cyclomethicone/dimethicone copolyol mixture sold under the name Q2-3225C® by Dow Corning;

c) anionic surfactants such as:
salts of C₁₆-C₃₀ fatty acids, in particular amino salts such as triethanolamine stearate or 2-amino-2-methylpropane-1,3-diol stearate;
salts of polyoxyethylenated fatty acids, in particular amino salts or alkali metal salts, and mixtures thereof.
phosphoric esters and salts thereof, such as “DEA oleth-10 phosphate” (CRODAFOS N 10N from Croda) or monopotassium monocetyl phosphate (AMPHISOL K from Givaudan or ARLATONE MAP 160K from Uniqema);
sulphosuccinates, such as “disodium PEG-5 citrate lauryl sulphosuccinate” and “disodium ricinoleamido MEA sulphosuccinate”;
alkyl ether sulphates, such as sodium lauryl ether sulphate;
isethionates;
acylglutamates, such as “disodium hydrogenated tallow glutamate” (AMISOFT HS-21 R® sold by Ajinomoto), and mixtures thereof.

By way of examples of a cationic surfactant, mention may in particular be made of:
alkylimidazolidiniums, such as isostearyl ethyl-imidonium ethosulphate,
ammonium salts, such as (C₁₂₋₃₀ alkyl)tri(C₁₋₄ alkyl)ammonium halides, for instance N,N,N-trimethyl-1-docosanaminium chloride (or behentrimonium chloride).

The compositions according to the invention may also contain one or more amphoteric surfactants, for instance N-acyl amino acids, such as N-alkyl aminoacetates and disodium cocoamphodiacetate, and amine oxides, such as stearamine oxide, or else silicone surfactants, for instance dimethicone copolyol phosphates, such as that sold under the name PECOSIL PS 100® by Phoenix Chemical.

According to one particular embodiment of the invention, the emulsifying system may comprise at least one surfactant chosen from:
i) an alkali metal alkylphosphate of formula or phosphine oxide (R—O)ₙ—P═O(O⁻M)ₘ with R representing a linear or branched C₈-C₂₂ alkyl group such as cetyl, n being equal to 1, 2 or 3 and m being equal to 0, 1 or 2, with m+n being equal to 3 and M representing a hydrogen atom or an alkali or alkaline-earth metal, preferably n=1 and m=2, and M is an alkali metal, such as sodium or potassium;
ii) a polyethoxylated alcohol of formula R′—(OCH₂CH₂)ₚ—OH with R′ representing a linear or branched C₁-C₃₀, preferably C₈-C₂₄, or better still C₁₂-C₁₈ alkyl and particularly represents CH₃—(CH₂)₁₇— and p representing an integer inclusively between 2 and 30, preferably between 2 and 20; such as steareth-20, steareth-2;
iii) a glutamic acid salt of formula R—CONH—C(COO⁻M)-C₂H₄—COO-M′ with R representing a linear or branched C₈-C₂₂ alkyl group such as stearyl and M′ representing an alkali or alkaline-earth metal; and
iv) an alkyl glucoside obtained by condensation of glucose and of linear or branched C₈-C₂₂ fatty alcohols such as a cetyl and stearyl mixture known as cetylstearyl.

In particular, the emulsifying system may comprise at least one surfactant chosen from potassium cetyl phosphate, steareth-2, steareth-20 and mixtures thereof. As a variant or additionally, this emulsifying system may comprise at least one surfactant chosen from sodium stearoyl glutamate and cetylstearyl glucoside, and mixtures thereof.

According to the invention or according to one advantageous embodiment, according to the subject of the invention in question, the makeup composition comprises less than 1% by weight of triethanolamine relative to the total weight of the composition, preferably less than 0.5% or even less than 0.1%, or is free of triethanolamine.

Hydrophilic Film-Forming Polymer

The makeup composition may comprise, in addition to the particular polyurethane of the present invention, at least one additional film-forming polymer. This additional film-forming polymer may be lipophilic or hydrophilic.

The expression “film-forming polymer” is understood to mean a polymer that is capable, by itself or in the presence of an auxiliary agent which is able to form a film, of forming a macroscopically continuous film that adheres to the eyelashes, preferably a cohesive film and better still a film whose cohesion and mechanical properties are such that said film can be isolated and manipulated separately, for example when said film is made by casting on a non-stick surface, for instance a Teflon-coated or silicone-coated surface.

Generally, the solids content of the additional film-forming polymer in the composition may range from 0.1% to 40% by weight, preferably from 0.5% to 30% and better still from 1% to 10% by weight relative to the total weight of the composition.

Among the film-forming polymers that may be used in the composition of the present invention, mention may be made of synthetic polymers, of free-radical type or of polycondensate type, and polymers of natural origin, and mixtures thereof.

The hydrophilic film-forming polymer may be a water-soluble polymer or may be in dispersion in an aqueous medium.

As examples of water-soluble film-forming polymers, mention may be made of:
proteins, such as proteins of plant origin, such as wheat or soy protein; proteins of animal origin, such as keratins, for example keratin hydrolysates and sulphonic keratins;
cellulose polymers, such as hydroxyethyl cellulose, hydroxypropyl cellulose, methyl cellulose, ethyl hydroxyethyl cellulose, carboxymethyl cellulose and also quaternized cellulose derivatives;
acrylic polymers or copolymers, such as polyacrylates or polymethacrylates;
vinyl polymers, such as polyvinylpyrrolidones, copolymers of methyl vinyl ether and of malic anhydride, the copolymer of vinyl acetate and of crotonic acid, copolymers of vinylpyrrolidone and of vinyl acetate, copolymers of vinylpyrrolidone and of caprolactam, or polyvinyl alcohol;
anionic, cationic, amphoteric or nonionic chitin or chitosan polymers;
gum arabics, guar gum, xanthan derivatives or karaya gum;
alginates and carrageenans;
glycoaminoglycans, hyaluronic acid and its derivatives;
shellac resin, gum sandarac, dammar resins, elemi gums and copal resins;
deoxyribonucleic acid;
mucopolysaccharides such as chondroitin sulphates;
and mixtures thereof.

The film-forming polymer may also be present in the composition in the form of particles in dispersion in an aqueous phase, generally known under the name of latex or pseudolatex. The techniques for preparing these dispersions are well known to a person skilled in the art.

As an aqueous dispersion of a film-forming polymer, use may be made of the acrylic dispersions sold under the names NEOCRYL XK-90®, NEOCRYL A-1070®, NEOCRYL A-1090®, NEOCRYL BT-62®, NEOCRYL A-1079® and NEOCRYL A-523® by Avecia-Neoresins, DOW LATEX 432® by Dow Chemical, DAITOSOL 5000 AD® or DAITOSOL 5000 SJ® by Daito Kasey Kogyo; SYNTRAN 5760® by Interpolymer, Allianz Opt® by Rohm and Haas or else aqueous dispersions of polyurethane, sold under the names NEOREZ R-981® and NEOREZ R-974® by Avecia-Neoresins, AVALURE UR-405®, AVALURE UR-410®, Avalure UR-425®, AVALURE UR-450®, SANCURE 875®, AVALURE UR-445® and SANCURE 2060® by Noveon, IMPRANIL 85® by Bayer or AQUAMERE H-1511® by Hydromer; sulphopolyesters, sold under the trade name EASTMAN AQ® by Eastman Chemical Products, vinyl dispersions, such as MEXOMER PAM®, aqueous dispersions of polyvinyl acetate, such as “VINYBRAN®” from Nisshin Chemical or those sold by Union Carbide, aqueous dispersions of vinylpyrrolidone/dimethylaminopropyl methacrylamide/lauryldimethylpropylmethacrylamidoammonium chloride terpolymer, such as Styleze W from ISP, aqueous dispersions of polyurethane/polyacrylic hybrid polymers, such as those sold under the references “HYBRIDUR®” by Air Products or “DUROMER®” by National Starch, dispersions of core/shell type: for example those sold by Atofina under the KYNAR reference (core: fluorinated—shell: acrylic) or else those described in the document U.S. Pat. No. 5,188,899 (core: silica—shell: silicone), and mixtures thereof.

According to one particular embodiment, the composition of the invention may comprise as hydrophilic film-forming polymers at least the combination of a cationic polymer and an anionic polymer.

The cationic polymer may be chosen from quaternary cellulose ether derivatives, copolymers of cellulose with a water-soluble quaternary ammonium monomer, cyclopolymers, cationic polysaccharides, cationic silicone polymers, vinylpyrrolidone/dialkylaminoalkyl acrylate or methacrylate quaternized or non-quaternized copolymers, quaternary polymers of vinylpyrrolidone and of vinylimidazole, and polyaminoamines, and mixtures thereof.

Preferably, the cationic polymer is a hydroxy(C₁-C₄)alkyl cellulose comprising quaternary ammonium groups.

The anionic polymer is advantageously chosen from:

A) homopolymers or copolymers of acrylic or methacrylic acid or salts thereof, copolymers of acrylic acid and of acrylamide and salts thereof, and the sodium salts of polyhydroxycarboxylic acids such as the copolymers of acrylic acid and of acrylamide sold in the form of their sodium salt under the names RETEN® by Hercules, sodium polymethacrylate sold under the name DARVAN No. 7® by Vanderbilt, and the sodium salts of polyhydroxycarboxylic acids sold under the name HYDAGEN F® by Henkel;

B) copolymers of acrylic or methacrylic acids with a monoethylenic monomer such as ethylene, styrene, vinyl esters, acrylic or methacrylic acid esters, optionally grafted onto a polyalkylene glycol such as polyethylene glycol; copolymers of this type comprising in their chain an optionally N-alkylated and/or hydroxyalkylated acrylamide unit, copolymers of acrylic acid and of a C₁-C₄ alkyl methacrylate, and terpolymers of vinylpyrrolidone, of acrylic acid and of a C₁-C₂₀ alkyl methacrylate;

C) copolymers derived from crotonic acid, such as those comprising in their chain vinyl acetate or propionate units and optionally other monomers such as allylic or methallylic esters, a vinyl ether or vinyl ester of a linear or branched saturated carboxylic acid with a long hydrocarbon-based chain, such as those comprising at least 5 carbon atoms, these polymers possibly being grafted;

D) polymers derived from maleic, fumaric or itaconic acids or anhydrides with vinyl esters, vinyl ethers, vinyl halides, phenylvinyl derivatives or acrylic acid and esters thereof; copolymers of maleic, citraconic or itaconic anhydrides and of an allylic or methallylic ester optionally comprising an acrylamide or methacrylamide group, an α-olefin, acrylic or methacrylic esters, acrylic or methacrylic acids or vinylpyrrolidone in their chain, the anhydride functions being monoesterified or monoamidated;

E) polyacrylamides comprising carboxylate groups,

F) deoxyribonucleic acid;

G) copolymers of at least one dicarboxylic acid, of at least one diol and of at least one difunctional aromatic monomer bearing an —SO₃M group with M representing a hydrogen atom, an ammonium ion NH₄⁺ or a metal ion;
and mixtures thereof.

The anionic polymers that are most particularly preferred are chosen from non-crosslinked anionic polymers such as monoesterified methyl vinyl ether/maleic anhydride copolymers sold under the name GANTREZ ES 425 by ISP, the acrylic acid/ethyl acrylate/N-tert-butylacrylamide terpolymers sold under the name ULTRAHOLD STRONG by BASF, the copolymers of methacrylic acid and of methyl methacrylate sold under the name EUDRAGIT L by Rohm Pharma, the vinyl acetate/vinyl tert-butylbenzoate/crotonic acid terpolymers and the crotonic acid/vinyl acetate/vinyl neododecanoate terpolymers sold under the name RESIN 28-29-30 by National Starch, the copolymers of methacrylic acid and of ethyl acrylate sold under the name LUVIMER MAEX or MAE by BASF, the vinylpyrrolidone/acrylic acid/lauryl methacrylate terpolymers sold under the name ACRYLIDONE LM by ISP and the acrylic or methacrylic acid homopolymers sold, for example, under the name VERSICOL E 5 or the sodium polymethacrylate sold under the name DARVAN 7 by Vanderbilt, and mixtures thereof.

The anionic polymer is preferably a sodium polymethacrylate.

Hydrophilic Thickener

The composition according to the invention may comprise at least one hydrophilic thickener.

These thickeners may be used alone or in combination. These thickeners may be chosen especially from cellulose polymers and gums.

The expression “hydrophilic thickener” is understood to mean a thickener that is soluble or dispersible in water.

Hydrophilic thickeners that may be mentioned in particular include water-soluble or water-dispersible thickening polymers. These may be chosen especially from:
modified or unmodified carboxyvinyl polymers, such as the products sold under the name CARBOPOL (CTFA name: carbomer) by Goodrich;
homopolymers or copolymers of acrylic or methacrylic acids or the salts thereof and the esters thereof and in particular the products sold under the names VERSICOL F® or VERSICOL K® by Allied Colloid, ULTRAHOLD 8® by Ciba-Geigy, polyacrylates and polymethacrylates such as the products sold under the names LUBRAJEL and NORGEL by Guardian or under the name HISPAJEL by Hispano Chimica, and polyacrylic acids of SYNTHALEN K type;
polyacrylamides;
copolymers of acrylic acid and of acrylamide sold in the form of the sodium salt thereof under the names RETEN® by Hercules, the sodium polymethacrylate sold under the name DARVAN 7® by Vanderbilt, and the sodium salts of polyhydroxycarboxylic acids sold under the name HYDAGEN F® by Henkel;
optionally crosslinked and/or neutralized 2-acrylamido-2-methylpropanesulphonic acid polymers and copolymers, for instance poly(2-acrylamido-2-methylpropanesulphonic acid) sold by Clariant under the name HOSTACERIN AMPS (CTFA name: ammonium polyacryldimethyltauramide);
crosslinked anionic copolymers of acrylamide and of AMPS, which are in the form of a W/O emulsion, such as those sold under the name SEPIGEL 305 (CTFA name: Polyacrylamide/C13-14 Isoparaffin/Laureth-7) and under the name SIMULGEL 600 (CTFA name: Acrylamide/Sodium acryloyldimethyltaurate copolymer/Isohexadecane/Poly-sorbate 80) by SEPPIC;
polyacrylic acid/alkyl acrylate copolymers of PEMULEN type;
associative polymers, for instance PEG-150/stearyl alcohol/SMDI copolymer sold under the name ACULYN 46 by Rohm & Haas, or steareth-100/PEG-136/HDI copolymer sold under the name RHEOLATE FX 1100 by Elementis);
and mixtures thereof.

The hydrophilic thickener may be chosen from associative polymers. For the purposes of the present invention, the expression “associative polymer” is understood to mean any amphiphilic polymer comprising in its structure at least one fatty chain and at least one hydrophilic portion. The associative polymers in accordance with the present invention may be anionic, cationic, nonionic or amphoteric.

Among the associative anionic polymers that may be mentioned are those comprising at least one hydrophilic unit and at least one fatty-chain allyl ether unit, more particularly from those in which the hydrophilic unit is constituted of an ethylenic unsaturated anionic monomer, more particularly a vinylcarboxylic acid and most particularly an acrylic acid, a methacrylic acid or mixtures thereof, and in which the fatty-chain allyl ether unit corresponds to the monomer of formula (I) below:

CH₂═C(R′)CH₂OBₙR (I)

in which R′ denotes H or CH₃, B denotes an ethyleneoxy radical, n is zero or denotes an integer ranging from 1 to 100, and R denotes a hydrocarbon-based radical chosen from alkyl, arylalkyl, aryl, alkylaryl and cycloalkyl radicals containing from 8 to 30 carbon atoms, preferably 10 to 24 and more particularly still from 12 to 18 carbon atoms.

Anionic amphiphilic polymers of this type are described and prepared, according to an emulsion polymerization process, in patent EP-0 216 479.

Associative anionic polymers that may also be mentioned include anionic polymers comprising at least one hydrophilic unit of olefinic unsaturated carboxylic acid type, and at least one hydrophobic unit exclusively of (C₁₀-C₃₀)alkyl ester of unsaturated carboxylic acid type. Examples that may be mentioned include the anionic polymers described and prepared according to U.S. Pat. Nos. 3,915,921 and 4 509 949.

Cationic associative polymers that may be mentioned include quaternized cellulose derivatives and polyacrylates containing amine side groups.

The nonionic associative polymers may be chosen from:
celluloses modified with groups comprising at least one fatty chain, for instance hydroxyethyl celluloses modified with groups comprising at least one fatty chain, such as alkyl groups, especially C₈-C₂₂ alkyl groups, arylalkyl and alkylaryl groups, such as NATROSOL PLUS GRADE 330 CS(C₁₆ alkyls) sold by Aqualon,
celluloses modified with polyalkylene glycol alkylphenyl ether groups,
guars such as hydroxypropyl guar, modified with groups comprising at least one fatty chain such as an alkyl chain,
copolymers of vinylpyrrolidone and of fatty-chain hydrophobic monomers,
copolymers of C₁-C₆ alkyl methacrylates or acrylates and of amphiphilic monomers comprising at least one fatty chain,
copolymers of hydrophilic methacrylates or acrylates and of hydrophobic monomers comprising at least one fatty chain, for instance the polyethylene glycol methacrylate/lauryl methacrylate copolymer, and
associative polyurethanes.

Associative polyurethanes are nonionic block copolymers comprising in the chain both hydrophilic blocks usually of polyoxyethylene nature, and hydrophobic blocks that may be aliphatic sequences alone and/or cycloaliphatic and/or aromatic sequences.

In particular, these polymers comprise at least two hydrocarbon-based lipophilic chains containing from C₆ to C₃₀ carbon atoms, separated by a hydrophilic block, the hydrocarbon-based chains possibly being pendent chains or chains at the end of a hydrophilic block. In particular, it is possible for one or more pendent chains to be provided. In addition, the polymer may comprise a hydrocarbon-based chain at one or both ends of a hydrophilic block. The associative polyurethanes may be arranged in triblock or multiblock form. The hydrophobic blocks may thus be at each end of the chain (for example: triblock copolymer with a hydrophilic central block) or distributed both at the ends and within the chain (for example multiblock copolymer). These polymers may also be graft polymers or starburst polymers. Preferably, the associative polyurethanes are triblock copolymers in which the hydrophilic block is a polyoxyethylene chain containing from 50 to 1000 oxyethylene groups. In general, the associative polyurethanes comprise a urethane bond between the hydrophilic blocks, whence the name.

By way of example, among the associative polymers that may be used in the invention, mention may be made of the polymer C₁₆-OE₁₂₀-C₁₅ from Servo Delden (under the name SER AD FX1100, which is a molecule containing a urethane function and having a weight-average molecular weight of 1300), OE being an oxyethylene unit. An associative polymer that may also be used is RHEOLATE 205 containing a urea function, sold by Rheox, or RHEOLATE 208 or 204 or alternatively RHEOLATE FX1100 from Elementis. These associative polyurethanes are sold in pure form. The product DW 1206B from Rohm & Haas containing a C₂₀ alkyl chain with a urethane bond, sold at a solids content of 20% in water, may also be used.

It is also possible to use solutions or dispersions of these polymers, especially in water or in aqueous-alcoholic medium. Examples of such polymers that may be mentioned include SER AD FX1010, SER AD FX1035 and SER AD 1070 from Servo Delden, and RHEOLATE 255, RHEOLATE 278 and RHEOLATE 244 sold by Rheox. It is also possible to use the product ACULYN 46, DW 1206F and DW 1206J, and also ACRYSOL RM 184 or ACRYSOL 44 from Rohm & Haas, or alternatively BORCHIGEL LW 44 from Borchers, and mixtures thereof.

According to one advantageous embodiment, the hydrophilic thickening polymer used in the composition of the invention is chosen from the copolymers resulting from the polymerization of at least one monomer (a) chosen from carboxylic acids possessing α,β-ethylenically unsaturated groups or their esters with at least one monomer (b) possessing ethylenically unsaturated groups and comprising a hydrophobic group.

This polymer may also exhibit emulsifying properties.

The thickening polymer is preferably anionic.

The term “copolymers” is understood to mean both copolymers obtained from two types of monomers and those obtained from more than two types of monomers, such as terpolymers obtained from three types of monomers.

Their chemical structure more particularly comprises at least one hydrophilic unit and at least one hydrophobic unit. The expression “hydrophobic group” or “hydrophobic unit” is understood to mean a radical possessing a saturated or unsaturated and linear or branched hydrocarbon-based chain which comprises at least 8 carbon atoms, preferably from 10 to 30 carbon atoms, in particular from 12 to 30 carbon atoms and more preferably from 18 to 30 carbon atoms.

Preferably, the thickening copolymers are chosen from the copolymers resulting from the polymerization:
of at least one monomer of formula (1) below:

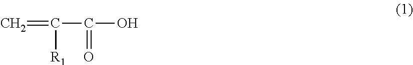

in which R₁ denotes H or CH₃ or C₂H₅, that is to say acrylic acid, methacrylic acid or ethacrylic acid monomers, and
of at least one monomer of (C₁₀-C₃₀) alkylester of unsaturated carboxylic acid type corresponding to the monomer of formula (2) below:

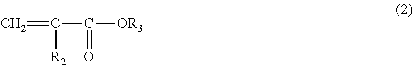

in which R₂ denotes H or CH₃ or C₂H₅ (that is to say, acrylate, methacrylate or ethacrylate units) and preferably H (acrylate units) or CH₃ (methacrylate units), R₃ denoting a C₁₀-C₃₀ and preferably C₁₂-C₂₂ alkyl radical.

The (C₁₀-C₃₀)alkyl esters of unsaturated carboxylic acids are preferably chosen from lauryl acrylate, stearyl acrylate, decyl acrylate, isodecyl acrylate, dodecyl acrylate and the corresponding methacrylates, such as lauryl methacrylate, stearyl methacrylate, decyl methacrylate, isodecyl methacrylate and dodecyl methacrylate, and mixtures thereof.

According to one preferred embodiment, these thickening polymers are crosslinked.

Use will more particularly be made, among thickening copolymers of this type, of polymers resulting from the polymerization of a mixture of monomers comprising:

(i) essentially acrylic acid,

(ii) an ester of formula (2) described above in which R₂ denotes H or CH₃, R₃ denoting an alkyl radical having from 12 to 22 carbon atoms,

(iii) and a crosslinking agent, which is a well-known copolymerizable polyethylenic unsaturated monomer, such as diallyl phthalate, allyl (meth)acrylate, divinylbenzene, (poly)ethylene glycol dimethacrylate and methylenebisacrylamide.

Use will more particularly be made, among copolymers of this type, of those constituted of 95 to 60% by weight of acrylic acid (hydrophilic unit), 4 to 40% by weight of C₁₀-C₃₀ alkyl acrylate (hydrophobic unit) and 0 to 6% by weight of crosslinking polymerizable monomer, or else of those constituted of 98 to 96% by weight of acrylic acid (hydrophilic unit), 1 to 4% by weight of C₁₀-C₃₀ alkyl acrylate (hydrophobic unit) and of 0.1 to 0.6% by weight of crosslinking polymerizable monomer, such as those described above.

Preference is very particularly given according to the present invention, among the above said polymers, to acrylate/C₁₀-C₃₀ alkyl acrylate copolymers (INCI name: Acrylates/C10-30 Alkyl Acrylate Crosspolymer), such as the products sold by Lubrizol under the trade names PEMULEN TR1, PEMULEN TR2, CARBOPOL 1382 and CARBOPOL EDT 2020 and more preferably still to PEMULEN TR-2.

The hydrophilic thickening polymer may represent, as active material, from 0.01 to 10% by weight, preferably from 0.05 to 5% by weight, better still from 0.05 to 3% by weight and even better still from 0.05 to 1% by weight, with respect to the total weight of the composition.

Fatty Phase

The composition according to the invention may also comprise a fatty phase comprising at least one oil and/or at least one wax, and/or at least one lipophilic film-forming polymer and/or a fatty phase rheological agent.

Oils

The term “oil” is intended to mean a fatty substance that is liquid at ambient temperature and at atmospheric pressure.

Volatile Oil

The composition according to the invention may comprise at least one volatile oil.

The term “volatile oil” is understood to mean an oil (or nonaqueous medium) capable of evaporating on contact with the skin in less than one hour, at ambient temperature and at atmospheric pressure. The volatile oil is a volatile cosmetic oil which is liquid at ambient temperature, having in particular a non-zero vapour pressure, at ambient temperature and at atmospheric pressure, in particular having a vapour pressure ranging from 0.13 Pa to 40 000 Pa (10⁻³ to 300 mmHg), and preferably ranging from 1.3 Pa to 13 000 Pa (0.01 to 100 mmHg), and preferentially ranging from 1.3 Pa to 1300 Pa (0.01 to 10 mmHg).

This volatile oil may be a hydrocarbon-based volatile oil.

The hydrocarbon-based volatile oil may be chosen from hydrocarbon-based oils having from 7 to 16 carbon atoms. The hydrocarbon-based volatile oil may be present in the composition according to the invention in a content ranging from 0.1% to 90% by weight, preferably ranging from 1% to 70% by weight and preferentially ranging from 5% to 70% by weight, or even 5% to 50% by weight, relative to the total weight of the composition.

The composition according to the invention may contain one or more volatile branched alkane(s). The expression “one or more volatile branched alkane(s)” is understood to equally mean “one or more volatile branched alkane oil(s)”.

Mention may especially be made, as a hydrocarbon-based volatile oil having from 7 to 16 carbon atoms, of branched C₈-C₁₆ alkanes such as C₈-C₁₆ isoalkanes (also known as isoparaffins), isododecane, isodecane and isohexadecane, and, for example, the oils sold under the trade names ISOPARS or PERMETYLS, branched C₈-C₁₆ esters such as isohexyl neopentanoate, and mixtures thereof. Preferably, the hydrocarbon-based volatile oil having from 8 to 16 carbon atoms is chosen from isododecane, isodecane, isohexadecane and mixtures thereof, and in particular is isododecane.

The composition according to the invention may contain one or more volatile linear alkane(s). The expression “one or more volatile linear alkane(s)” is understood to equally mean “one or more volatile linear alkane oil(s)”.

A volatile linear alkane suitable for the invention is liquid at ambient temperature (around 25° C.) and at atmospheric pressure (760 mmHg).

The expression “volatile linear alkane”, suitable for the invention, is understood to mean a cosmetic linear alkane capable of evaporating on contact with the skin in less than one hour, at ambient temperature (25° C.) and atmospheric pressure (760 mmHg, that is to say 101 325 Pa), that is liquid at ambient temperature, and that has, in particular, an evaporation rate ranging from 0.01 to 15 mg/cm²/min at ambient temperature (25° C.) and atmospheric pressure (760 mmHg).

Preferably, the “volatile linear alkanes” suitable for the invention have an evaporation rate ranging from 0.01 to 3.5 mg/cm²/min at ambient temperature (25° C.) and atmospheric pressure (760 mmHg).

Preferably, the “volatile linear alkanes” suitable for the invention have an evaporation rate ranging from 0.01 to 1.5 mg/cm²/min at ambient temperature (25° C.) and atmospheric pressure (760 mmHg).

More preferably, the “volatile linear alkanes” suitable for the invention have an evaporation rate ranging from 0.01 to 0.8 mg/cm²/min at ambient temperature (25° C.) and atmospheric pressure (760 mmHg).

More preferably, the “volatile linear alkanes” suitable for the invention have an evaporation rate ranging from 0.01 to 0.3 mg/cm²/min at ambient temperature (25° C.) and atmospheric pressure (760 mmHg).

More preferably, the “volatile linear alkanes” suitable for the invention have an evaporation rate ranging from 0.01 to 0.12 mg/cm²/min at ambient temperature (25° C.) and atmospheric pressure (760 mmHg).

The evaporation rate of a volatile alkane according to the invention (and more generally of a volatile solvent) may especially be evaluated by means of the protocol described in WO 06/013413, and more particularly by means of the protocol described below.

Introduced into a crystallizing dish (diameter: 7 cm), placed on a balance that is located in a chamber of around 0.3 m³, the temperature (25° C.) and hygrometry (50% relative humidity) of which are regulated, are 15 g of volatile hydrocarbon-based solvent.

The liquid is left to evaporate freely, without being stirred, ventilation being provided by a fan (PAPST-MOTOREN, reference 8550 N, rotating at 2700 rpm) placed in a vertical position above the crystallizing dish containing the volatile hydrocarbon-based solvent, the blades being directed towards the crystallizing dish, at a distance of 20 cm relative to the base of the crystallizing dish.

The mass of volatile hydrocarbon-based solvent remaining in the crystallizing dish is measured at regular time intervals.

The evaporation profile of the solvent is then obtained by plotting the curve of the amount of product evaporated (in mg/cm²) as a function of the time (in min).

Then the evaporation rate, which corresponds to the tangent at the origin of the curve obtained, is calculated. The evaporation rates are expressed as mg of volatile solvent evaporated per unit area (cm²) and per unit time (minutes).

According to one preferred embodiment, the “volatile linear alkanes” suitable for the invention have a non-zero vapour pressure (also known as saturation vapour pressure) at ambient temperature, in particular a vapour pressure ranging from 0.3 Pa to 6000 Pa.

Preferably, the “volatile linear alkanes” suitable for the invention have a vapour pressure ranging from 0.3 to 2000 Pa at ambient temperature (25° C.).

Preferably, the “volatile linear alkanes” suitable for the invention have a vapour pressure ranging from 0.3 to 1000 Pa at ambient temperature (25° C.).

More preferably, the “volatile linear alkanes” suitable for the invention have a vapour pressure ranging from 0.4 to 600 Pa at ambient temperature (25° C.).

Preferably, the “volatile linear alkanes” suitable for the invention have a vapour pressure ranging from 1 to 200 Pa at ambient temperature (25° C.).

More preferably, the “volatile linear alkanes” suitable for the invention have a vapour pressure ranging from 3 to 60 Pa at ambient temperature (25° C.).

According to one embodiment, a linear volatile alkane suitable for the invention may have a flashpoint in the range that varies from 30 to 120° C., and more particularly from 40 to 100° C. The flashpoint is, in particular, measured according to the ISO 3679 standard.

According to one embodiment, an alkane suitable for the invention may be a volatile linear alkane comprising from 7 to 14 carbon atoms.

Preferably, the “volatile linear alkanes” suitable for the invention comprise from 8 to 14 carbon atoms.

Preferably, the “volatile linear alkanes” suitable for the invention comprise from 9 to 14 carbon atoms.

Preferably, the “volatile linear alkanes” suitable for the invention comprise from 10 to 14 carbon atoms.

Preferably, the “volatile linear alkanes” suitable for the invention comprise from 11 to 14 carbon atoms.

According to one advantageous embodiment, the “volatile linear alkanes” suitable for the invention have an evaporation rate, as defined above, ranging from 0.01 to 3.5 mg/cm²/min at ambient temperature (25° C.) and atmospheric pressure (760 mmHg) and comprise from 8 to 14 carbon atoms.

A volatile linear alkane suitable for the invention may advantageously be of plant origin.

Preferably, the volatile linear alkane or the mixture of volatile linear alkanes present in the composition according to the invention comprises at least one ¹⁴C isotope of carbon (carbon-14), in particular the ¹⁴C isotope may be present in a ¹⁴C/¹²C ratio greater than or equal to 1×10⁻¹⁶, preferably greater than or equal to 1×10⁻¹⁵, more preferably greater than or equal to 7.5×10⁻¹⁴, and better still greater than or equal to 1.5×10⁻¹³. Preferably, the ¹⁴C/¹²C ratio ranges from 6×10⁻¹³ to 1.2×10⁻¹².

The amount of ¹⁴C isotopes in the volatile linear alkane or the mixture of volatile linear alkanes may be determined by methods known to a person skilled in the art such as the Libby counting method, liquid scintillation spectrometry or else accelerator mass spectrometry.

Such an alkane may be obtained, directly or in several steps, from a plant raw material such as an oil, a butter, a wax, etc.

As examples of alkanes suitable for the invention, mention may be made of the alkanes described in patent application WO 2007/068371 or WO 2008/155059 by Cognis (mixtures of different alkanes that differ by at least one carbon). These alkanes are obtained from fatty alcohols that are themselves obtained from coconut oil or palm oil.

By way of example of a linear alkane suitable for the invention, mention may be made of n-heptane (C₇), n-octane (C₈), n-nonane (C₉), n-decane (C₁₀), n-undecane (C₁₁), n-dodecane (C₁₂), n-tridecane (C₁₃), n-tetradecane (C₁₄) and mixtures thereof. According to one particular embodiment, the volatile linear alkane is chosen from n-nonane, n-undecane, n-dodecane, n-tridecane, n-tetradecane, and mixtures thereof.

According to one preferred mode, mention may be made of the mixtures of n-undecane (C₁₁) and of n-tridecane (C₁₃) obtained in examples 1 and 2 of application WO 2008/155059 by Cognis.

Mention may also be made of n-dodecane (C₁₂) and n-tetradecane (C₁₄) sold by Sasol respectively under the references PARAFOL 12-97 and PARAFOL 14-97, and also mixtures thereof.

The volatile linear alkane could be used alone.

Alternatively or preferably a mixture of at least two different volatile linear alkanes could be used, that differ from one another by a carbon number n of at least 1, in particular that differ from one another by a carbon number of 1 or 2.

According to a first embodiment, use is made of a mixture of at least two different volatile linear alkanes comprising from 10 to 14 carbon atoms that differ from one another by a carbon number of at least 1. By way of examples, mention may especially be made of the C₁₀/C₁₁, C₁₁/C₁₂ or C₁₂/C₁₃ mixtures of volatile linear alkanes.

According to another embodiment, use is made of a mixture of at least two different volatile linear alkanes comprising from 10 to 14 carbon atoms that differ from one another by a carbon number of at least 2. By way of examples, mention may especially be made of the C₁₀/C₁₂ or C₁₂/C₁₄ mixtures of volatile linear alkanes, for an even carbon number n and the C₁₁/C₁₃ mixture for an odd carbon number n.

According to one preferred mode, use is made of a mixture of at least two different volatile linear alkanes comprising from 10 to 14 carbon atoms that differ from one another by a carbon number of at least 2, and in particular a C₁₁/C₁₃ mixture of volatile linear alkanes or a C₁₂/C₂₄ mixture of volatile linear alkanes.

Other mixtures combining more than 2 volatile linear alkanes according to the invention, such as for example a mixture of at least 3 different volatile linear alkanes comprising from 7 to 14 carbon atoms that differ from one another by a carbon number of at least 1, are also part of the invention, but the mixtures with 2 volatile linear alkanes according to the invention are preferred (binary mixtures), said 2 volatile linear alkanes preferably representing more than 95% and better still more than 99% by weight of the total content of volatile linear alkanes in the mixture. According to one particular mode of the invention, in a mixture of volatile linear alkanes, the volatile linear alkane having the smallest carbon number is predominant in the mixture.

According to another mode of the invention, use is made of a mixture of volatile linear alkanes in which the volatile linear alkane having the highest carbon number is predominant in the mixture.

By way of examples of mixtures suitable for the invention, mention may especially be made of the following mixtures:
from 50 to 90% by weight, preferably from 55 to 80% by weight, more preferably from 60 to 75% by weight of Cₙ volatile linear alkane with n ranging from 7 to 14;
from 10 to 50% by weight, preferably from 20 to 45% by weight, preferably from 24 to 40% by weight of Cₙ₊ₓ volatile linear alkane with x greater than or equal to 1, preferably x=1 or x=2, with n+x between 8 and 14,
   relative to the total weight of alkanes in said mixture.

In particular, said mixture of alkanes according to the invention contains:
less than 2% by weight, preferably less than 1% by weight, of branched hydrocarbons;
and/or less than 2% by weight, preferably less than 1% by weight, of aromatic hydrocarbons;
and/or less than 2% by weight, preferably less than 1% by weight and preferentially less than 0.1% by weight, of unsaturated hydrocarbons in the mixture.

More particularly, a volatile linear alkane suitable for the invention may be used in the form of an n-undecane/n-tridecane mixture.

In particular, use will be made of a mixture of volatile linear alkanes comprising:
from 55 to 80% by weight, preferably from 60 to 75% by weight, of C₁₁ (n-undecane) volatile linear alkane;
from 20 to 45% by weight, preferably from 24 to 40% by weight, of C₁₃ (n-tridecane) volatile linear alkane,
   relative to the total weight of alkanes in said mixture.

According to one particular embodiment, the mixture of alkanes is an n-undecane/n-tridecane mixture. In particular, such a mixture may be obtained according to Example 1 or Example 2 of WO 2008/155059.

According to another particular embodiment, use is made of the n-dodecane sold under the reference PARAFOL 12-97 by Sasol.

According to another particular embodiment, use is made of the n-tetradecane sold under the reference PARAFOL 14-97 by Sasol.

According to yet another embodiment, use is made of a mixture of n-dodecane and of n-tetradecane.

As a variant or additionally, the composition produced may comprise at least one volatile silicone solvent or oil, which is compatible with a cosmetic use.

The term “silicone oil” is understood to mean an oil that contains at least one silicon atom, and in particular that contains Si—O groups. According to one embodiment, said composition comprises less than 10% by weight of non-volatile silicone oils, relative to the total weight of the composition, better still less than 5% by weight, or even is free of silicone oil.

Mention may be made, as silicone volatile oil, of cyclic polysiloxanes, linear polysiloxanes and mixtures thereof. As linear volatile polysiloxanes, mention may be made of hexamethyldisiloxane, octamethyltrisiloxane, decamethyltetrasiloxane, tetradecamethylhexasiloxane and hexadecamethylheptasiloxane. As cyclic volatile polysiloxanes, mention may be made of hexamethylcyclo-trisiloxane, octamethylcyclotetrasiloxane, decamethyl-cyclopentasiloxane and dodecamethylcyclohexasiloxane.

As a variant or additionally, the composition produced may comprise at least one volatile fluoro oil.

The term “fluoro oil” is understood to mean an oil containing at least one fluorine atom.

Mention may be made, as a volatile fluoro oil, of nonafluoromethoxybutane or perfluoromethylcyclopentane, and mixtures thereof.

The volatile oil may be present in a content ranging from 0.1% to 90% by weight, preferably ranging from 1% to 70% by weight and preferentially ranging from 5% to 50% by weight, relative to the total weight of the composition.

Non-Volatile Oil

The composition according to the invention may comprise at least one non-volatile oil.

The oil may be selected from hydrocarbon-based, fluoro and/or silicone oils.

Non-volatile hydrocarbon-based oils that may be used include liquid paraffin (or petroleum jelly), squalane, hydrogenated polyisobutylene (Parleam oil), perhydrosqualene, mink oil, turtle oil, soybean oil, sweet almond oil, beauty-leaf oil, palm oil, grapeseed oil, sesame oil, corn oil, arara oil, rapeseed oil, sunflower oil, cotton oil, apricot oil, castor oil, avocado oil, jojoba oil, olive oil or cereal germ oil; esters of lanolic acid, of oleic acid, of lauric acid or of stearic acid; fatty esters, especially of C₁₂-C₃₆, such as isopropyl myristate, isopropyl palmitate, butyl stearate, hexyl laurate, diisopropyl adipate, isononyl isononanoate, 2-ethylhexyl palmitate, 2-hexyldecyl laurate, 2-octyldecyl palmitate, 2-octyldodecyl myristate or lactate, bis(2-ethylhexyl)succinate, diisostearyl malate, and glyceryl or diglyceryl triisostearate; behenic acid, oleic acid, linoleic acid, linolenic acid or isostearic acid; higher fatty alcohols, especially of C₁₆-C₂₂, such as cetanol, oleyl alcohol, linoleyl alcohol or linolenyl alcohol, isostearyl alcohol or octyldodecanol; and mixtures thereof.

The fluoro oils which can be used in the invention are in particular fluorosilicone oils, fluorinated polyethers or fluorinated silicones, such as described in the document EP-A-847 752.

The non-volatile oil may be present in a content ranging from 0.1 to 70% by weight, preferably ranging from 0.5% to 60% by weight and preferentially ranging from 1% to 50% by weight, relative to the total weight of the composition.

Waxes

The composition may comprise at least one wax.

For the purposes of the present invention, the term “wax” is understood to mean a lipophilic compound, which is solid at room temperature (25° C.), with a reversible solid/liquid change of state, which has a melting point of greater than or equal to 30° C., which may range up to 120° C.

The melting point of the wax may be measured using a differential scanning calorimeter (DSC), for example the calorimeter sold under the name DSC 30 by Mettler.

The waxes may be hydrocarbon-based waxes, fluoro waxes and/or silicone waxes and may be of plant, mineral, animal and/or synthetic origin. In particular, the waxes have a melting point of greater than 25° C. and better still greater than 45° C.

The wax may be present in a content at least equal to 15% by weight. Preferably, it is present in a content ranging from 15% to 40% by weight, better still from 16% to 35% and even better still from 16% to 30% by weight relative to the total weight of the composition. The wax may be present in the composition in a content ranging from 0.1% to 50% by weight, preferably ranging from 1% to 40% and preferentially ranging from 5% to 30% by weight relative to the total weight of the composition.

Hydrocarbon-based waxes, for instance beeswax, lanolin wax or Chinese insect wax; rice wax, carnauba wax, candelilla wax, ouricury wax, esparto grass wax, cork fibre wax, sugarcane wax, Japan wax and sumach wax; montan wax, microcrystalline waxes, paraffins and ozokerite; polyethylene waxes, the waxes obtained by Fisher-Tropsch synthesis and waxy copolymers, and also esters thereof, may especially be used.

Mention may also be made of waxes obtained by catalytic hydrogenation of animal or plant oils containing linear or branched C₈-C₃₂ fatty chains.

Among these waxes, mention may especially be made of hydrogenated jojoba oil, isomerized jojoba oil such as the trans-isomerized partially hydrogenated jojoba oil manufactured or sold by Desert Whale under the commercial reference ISO-JOJOBA-50®, hydrogenated sunflower oil, hydrogenated castor oil, hydrogenated coconut oil, hydrogenated lanolin oil, bis(1,1,1-trimethylolpropane) tetrastearate sold under the name HEST 2T-4S by Heterene and bis(1,1,1-trimethylol-propane) tetrabehenate sold under the name HEST 2T-4B by Heterene.

Mention may also be made of silicone waxes, for instance alkyl or alkoxy dimeticones containing from 16 to 45 carbon atoms, and fluoro waxes.

The wax obtained by hydrogenation of olive oil esterified with stearyl alcohol, sold under the name PHYTOWAX OLIVE 18L57 or else the waxes obtained by hydrogenation of castor oil esterified with cetyl alcohol sold under the names PHYTOWAX RICIN 16L64 and 22L73 by Sophim may also be used. Such waxes are described in patent application FR-A-2 792 190.

The composition may comprise at least one polar wax. The expression “polar wax” is understood to mean waxes comprising in their chemical structure, in addition to carbon and hydrogen atoms, at least one highly electronegative heteroatom, such as O, N or P.

Preferably, the wax is chosen from carnauba wax, candelilla wax, natural (or bleached) beeswax, and synthetic beeswax. As synthetic beeswax, mention may be made of the wax sold under the name CYCLOCHEM 326 A by Evonik Goldschmidt (INCI name: Synthetic Beeswax).

The composition may comprise at least one wax having a hardness ranging from 0.05 MPa to 15 MPa, and preferably ranging from 6 MPa to 15 MPa. The hardness is determined by measuring the compression force, which is measured at 20° C. using the texturometer sold under the name TA-TX21 by Rheo, equipped with a stainless-steel cylinder 2 mm in diameter, travelling at a measuring speed of 0.1 mm/s, and penetrating into the wax to a penetration depth of 0.3 mm.

According to one particular embodiment, the compositions according to the invention may comprise at least one wax referred to as a tacky wax, i.e. a wax with a tack of greater than or equal to 0.7 N.s and a hardness of less than or equal to 3.5 MPa

Using a tacky wax may especially make it possible to obtain a cosmetic composition that applies easily to the eyelashes, attaches well to the eyelashes and leads to the formation of a smooth, uniform and thickening makeup result.

The tacky wax used may especially have a tack ranging from 0.7 N.s to 30 N.s, in particular greater than or equal to 1 N.s, especially ranging from 1 N.s to 20 N.s, in particular greater than or equal to 2N.s, especially ranging from 2 N.s to 10 N.s and in particular ranging from 2 N.s to 5 N.s.

The tack of the wax is determined by measuring the change in force (compression force or stretching force) as a function of time, at 20° C., using the texturometer sold under the name TA-TX2i® by Rheo, equipped with a conical acrylic polymer spindle forming an angle of 45°.

The measuring protocol is as follows:

The wax is melted at a temperature equal to the melting point of the wax+10° C. The molten wax is poured into a container 25 mm in diameter and 20 mm deep. The wax is recrystallized at room temperature (25° C.) for 24 hours such that the surface of the wax is flat and smooth, and the wax is then stored for at least 1 hour at 20° C. before measuring the tack.

The texturometer spindle is displaced at a speed of 0.5 mm/s then penetrates the wax to a penetration depth of 2 mm. When the spindle has penetrated the wax to a depth of 2 mm, the spindle is held still for 1 second (corresponding to the relaxation time) and is then withdrawn at a speed of 0.5 mm/s.

During the relaxation time, the force (compression force) decreases greatly until it becomes zero, and then, during the withdrawal of the spindle, the force (stretching force) becomes negative and then rises again to the value 0. The tack corresponds to the integral of the curve of the force as a function of time for the part of the curve corresponding to negative values of the force (stretching force). The tack value is expressed in N.s.

The tacky wax that may be used generally has a hardness of less than or equal to 3.5 MPa, in particular ranging from 0.01 MPa to 3.5 MPa, especially ranging from 0.05 MPa to 3 MPa or even ranging from 0.1 MPa to 2.5 MPa.

The hardness is measured according to the protocol described previously.

Tacky waxes that may be used include a C₂₀-C₄₀ alkyl (hydroxystearyloxy)stearate (the alkyl group comprising from 20 to 40 carbon atoms), alone or as a mixture, in particular a C₂₀-C₄₀ alkyl 12-(12′-hydroxystearyloxy)stearate, of formula (II):

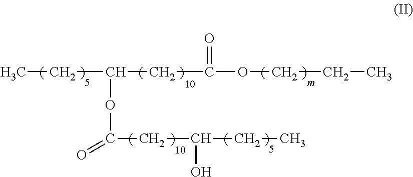

in which m is an integer ranging from 18 to 38, or a mixture of compounds of formula (II).

Such a wax is especially sold under the names KESTER WAX K 82 P® and KESTER WAX K 80 P® by Koster Keunen.

The waxes mentioned above generally have a starting melting point of less than 45° C.

Use may also be made of the microcrystalline wax sold under the reference SP18 by Strahl and Pitsch, which has a hardness of around 0.46 MPa and a tack value of around 1 N.s.

The wax(es) may be present in the form of an aqueous microdispersion of wax. The expression “aqueous microdispersion of wax” is understood to mean an aqueous dispersion of wax particles in which the size of said wax particles is less than or equal to about 1 μm.

Wax microdispersions are stable dispersions of colloidal wax particles, and are described especially in “Microemulsions Theory and Practice”, L. M. Prince Ed., Academic Press (1977) pages 21-32.

In particular, these wax microdispersions may be obtained by melting the wax in the presence of a surfactant, and optionally of a portion of water, followed by gradual addition of hot water with stirring. The intermediate formation of an emulsion of the water-in-oil type is observed, followed by a phase inversion, with final production of a microemulsion of the oil-in-water type. On cooling, a stable microdispersion of solid colloidal wax particles is obtained.

The wax microdispersions may also be obtained by stirring the mixture of wax, surfactant and water using stirring means such as ultrasound, high-pressure homogenizers or turbomixers.

The particles of the wax microdispersion preferably have mean sizes of less than 1 μm (especially ranging from 0.02 μm to 0.99 μm) and preferably less than 0.5 μm (especially ranging from 0.06 μm to 0.5 μm).

These particles are essentially constituted of a wax or a mixture of waxes. However, they may comprise a small proportion of oily and/or pasty fatty additives, a surfactant and/or a common liposoluble additive/active agent.

Lipophilic Film-Forming Polymer

The composition according to the invention may comprise at least one lipophilic film-forming polymer, which may be liposoluble (i.e. soluble in a liquid fatty phase comprising oils or organic solvents such as those described above) or may be present in the composition in the form of particles in dispersion in a non-aqueous solvent phase with which it is compatible, which may be the oily phase of the composition according to the invention.

As examples of liposoluble polymers, mention may be made of copolymers of vinyl ester (the vinyl group being directly linked to the oxygen atom of the ester group and the vinyl ester containing a saturated, linear or branched hydrocarbon-based radical of 1 to 19 carbon atoms, linked to the carbonyl of the ester group) and of at least one other monomer, which may be a vinyl ester (other than the vinyl ester already present), an α-olefin (containing from 8 to 28 carbon atoms), an alkyl vinyl ether (in which the alkyl group comprises from 2 to 18 carbon atoms) or an allylic or methallylic ester (containing a saturated, linear or branched hydrocarbon-based radical of 1 to 19 carbon atoms, linked to the carbonyl of the ester group).

These copolymers may be crosslinked with the aid of crosslinking agents, which may be either of the vinyl type or of the allylic or methallylic type, such as tetraallyloxyethane, divinylbenzene, divinyl octanedioate, divinyl dodecanedioate and divinyl octadecanedioate.

As examples of these copolymers, mention may be made of the following copolymers: vinyl acetate/allyl stearate, vinyl acetate/vinyl laurate, vinyl acetate/vinyl stearate, vinyl acetate/octadecene, vinyl acetate/octadecyl vinyl ether, vinyl propionate/allyl laurate, vinyl propionate/vinyl laurate, vinyl stearate/1-octadecene, vinyl acetate/1-dodecene, vinyl stearate/ethyl vinyl ether, vinyl propionate/cetyl vinyl ether, vinyl stearate/allyl acetate, vinyl 2,2-dimethyloctanoate/vinyl laurate, allyl 2,2-dimethylpentanoate/vinyl laurate, vinyl dimethyl-propionate/vinyl stearate, allyl dimethylpropionate/vinyl stearate, vinyl propionate/vinyl stearate, crosslinked with 0.2% divinylbenzene, vinyl dimethyl-propionate/vinyl laurate, crosslinked with 0.2% divinylbenzene, vinyl acetate/octadecyl vinyl ether, crosslinked with 0.2% tetraallyloxyethane, vinyl acetate/allyl stearate, crosslinked with 0.2% divinylbenzene, vinyl acetate/1-octadecene, crosslinked with 0.2% divinylbenzene, and allyl propionate/allyl stearate, crosslinked with 0.2% divinylbenzene.

As examples of liposoluble film-forming polymers, mention may also be made of liposoluble copolymers, and in particular those resulting from the copolymerization of vinyl esters containing from 9 to 22 carbon atoms or of alkyl acrylates or methacrylates, the alkyl radicals containing from 10 to 20 carbon atoms.

Such liposoluble copolymers may be chosen from copolymers of polyvinyl stearate, and of polyvinyl stearate crosslinked using divinylbenzene, diallyl ether or diallyl phthalate, copolymers of polystearyl (meth)acrylate, of polyvinyl laurate and of polylauryl (meth)acrylate, it being possible for these poly(meth)acrylates to be crosslinked using ethylene glycol dimethacrylate or tetraethylene glycol dimethacrylate.

The liposoluble copolymers defined above are known and are described in particular in patent application FR-A-2 232 303; they may have a weight-average molecular weight ranging from 2000 to 500 000 and preferably from 4000 to 200 000.

As liposoluble film-forming polymers that may be used in the invention, mention may also be made of polyalkylenes and in particular copolymers of C₂-C₂₀ alkenes, such as polybutene, alkyl celluloses with a linear or branched, saturated or unsaturated C₁-C₈ alkyl radical, for instance ethyl cellulose and propyl cellulose, copolymers of vinylpyrrolidone (VP) and in particular copolymers of vinylpyrrolidone and of C₂ to C₄₀ and better still C₃ to C₂₀ alkene. As examples of VP copolymers which may be used in the invention, mention may be made of the VP/vinyl acetate, VP/ethyl methacrylate, butylated polyvinylpyrrolidone (PVP), VP/ethyl methacrylate/methacrylic acid, VP/eicosene, VP/hexadecene, VP/triacontene, VP/styrene or VP/acrylic acid/lauryl methacrylate copolymers.

The lipophilic film-forming polymer may be chosen from the vinyl acetate/allyl stearate copolymer (sold in particular under the name MEXOMERE PQ by Chimex), the polyvinyl laurate (sold in particular under the name MEXOMERE PP by Chimex), the vinylpyrrolidone/eicosene copolymer (sold in particular under the name ANTARON V 220 by ISP), and mixtures thereof.

Mention may also be made of silicone resins, which are generally soluble or swellable in silicone oils, which are crosslinked polyorganosiloxane polymers. The nomenclature of silicone resins is known under the name “MDTQ”, the resin being described as a function of the various siloxane monomer units it comprises, each of the letters “MDTQ” characterizing a type of unit.

As examples of commercially available polymethyl-silsesquioxane resins, mention may be made of those sold by Wacker under the reference RESIN MK, such as BELSIL PMS MK, or by Shin-Etsu under the reference KR-220L.

As examples of commercially available polypropyl-silsesquioxane resins, mention may be made of those sold under the reference DC670 by Dow Corning.

As siloxysilicate resins, mention may be made of trimethylsiloxysilicate (TMS) resins such as those sold under the reference SR 1000 by General Electric or under the reference TMS 803 by Wacker. Mention may also be made of the trimethylsiloxysilicate resins sold in a solvent such as cyclomethicone, sold under the name KF-7312J by Shin-Etsu, and DC 749 and DC 593 by Dow Corning.

Mention may also be made of copolymers of silicone resins such as those mentioned above with polydimethylsiloxanes, for instance the pressure-sensitive adhesive copolymers sold by Dow Corning under the reference BIO-PSA and described in document U.S. Pat. No. 5,162,410, or else the silicone copolymers derived from the reaction of a silicone resin, such as those described above, and a diorganosiloxane as described in document WO 2004/073626.

The lipophilic film-forming polymer may also be a vinyl polymer comprising at least one carbosiloxane dendrimer-based unit.

Particularly suitable for the invention are the vinyl polymers comprising carbosiloxane dendrimer-based units such as, for example, those described in documents WO 2006/058793 and EP 1 862 162.

The vinyl polymer may especially have a backbone and at least one side chain, which comprises a carbosiloxane dendrimer structure. The expression “carbosiloxane dendrimer structure”, in the context of the present invention, represents a molecular structure with branched groups of high molecular masses, said structure having high regularity in the radial direction starting from the backbone bond. Such carbosiloxane dendrimer structures are described in the form of a highly branched siloxane-silylalkylene copolymer in the laid-open Japanese patent application Kokai 9-171 154.

The vinyl polymers grafted with at least one carbosiloxane dendrimer-based unit that may be very particularly suitable for the present invention are the polymers sold under the names TIB 4-100, TIB 4-101, TIB 4-120, TIB 4-130, TIB 4-200, FA 4002 ID (TIB 4-202), TIB 4-220 and FA 4001 CM (TIB 4-230) by Dow Corning.

Use may also be made of silicone-based polyamides of the polyorganosiloxane type such as those described in documents U.S. Pat. No. 5,874,069, U.S. Pat. No. 5,919,441, U.S. Pat. No. 6,051,216 and U.S. Pat. No. 5,981,680.

These silicone polymers may belong to the following two families:
polyorganosiloxanes comprising at least two groups capable of establishing hydrogen interactions, these two groups being located in the polymer chain; and/or
polyorganosiloxanes comprising at least two groups capable of establishing hydrogen interactions, these two groups being located on grafts or branchings.

The lipophilic or liposoluble film-forming polymer may also be present in the composition in the form of particles in dispersion in a non-aqueous solvent phase, which may be the oily phase of the composition. The techniques for preparing these dispersions are well known to those skilled in the art. For example, polymers of NAD (non-aqueous dispersion) type or microgels (for example KSG) may be used, and also styrene-based copolymers (Kraton, Regalite).

As examples of lipodispersible non-aqueous film-forming polymer dispersions in the form of non-aqueous dispersions of polymer particles in one or more silicone and/or hydrocarbon-based oils and which may be stabilized at their surface by at least one stabilizer, especially a block, graft or random polymer, mention may be made of the acrylic dispersions in isododecane, for instance MEXOMER PAP® from Chimex, dispersions of particles of a grafted ethylenic polymer, preferably an acrylic polymer, in a liquid fatty phase, the ethylenic polymer advantageously being dispersed in the absence of additional stabilizer at the surface of the particles, as described especially in document WO 04/055 081.

According to one embodiment example of the invention, the film-forming polymer is a film-forming linear block ethylenic polymer, which preferably comprises at least one first block and at least one second block that have different glass transition temperatures (T_{g}), said first and second blocks being joined to one another by an intermediate block comprising at least one monomer that is a constituent of the first block and at least one monomer that is a constituent of the second block.

Advantageously, the first and second blocks of the block polymer are incompatible with one another.

Such polymers are described, for example, in documents EP 1 411 069 or WO 04/028488.

As other examples of the film-forming system that can be used in the compositions according to the invention, mention may be made of the systems in which the film is formed in situ at the time of application of the composition or of a mixture of compositions containing two silicone compounds that react when they are brought into contact with one another. Such systems are described in particular in patent application WO 2007/071706. Systems of this type are also described in patent applications US 2007/142575 or US 2007/142599.

The lipophilic film-forming polymer may represent from 0.1% to 15%, preferably from 0.5% to 10%, by weight relative to the total weight of the composition.

Fatty-Phase Rheological Agent

The composition according to the invention may comprise a liquid fatty-phase rheological agent chosen from pasty compounds, semicrystalline polymers, lipophilic gelling agents and mixtures thereof.

The expression “fatty-phase rheological agent” is understood to mean an agent capable of establishing physical interactions, where necessary in contact with a crosslinking agent, when the structuring agent is not crosslinked, in said fatty phase within which it is useful. It has the ability to develop structuring properties—for example gelling properties—and thus results in textures having a semi-solid appearance.

The fatty-phase rheological agent may represent from 0.1 to 60%, preferably from 0.5 to 50% and more preferably still from 1 to 40% by weight, relative to the total weight of the composition.

Pasty Compounds

The expression “pasty compound” or “pasty fatty substance” is understood to mean a lipophilic fatty compound comprising, at a temperature of 23° C., a liquid fraction and a solid fraction.

Said pasty compound preferably has a hardness, at 20° C., ranging from 0.001 to 0.5 MPa, preferably from 0.002 to 0.4 MPa.

The hardness is measured according to a method of penetration of a probe into a sample of compound and in particular using a texture analyser (for example, the TA-XT2i from Rheo) equipped with a stainless steel cylinder with a diameter of 2 mm. The hardness measurement is carried out at 20° C. at the centre of 5 samples. The cylinder is introduced into each sample at a pre-rate of 1 mm/s and then at a measuring rate of 0.1 mm/s, the depth of penetration being 0.3 mm. The value recorded for the hardness is that of the maximum peak.

The liquid fraction of the pasty compound measured at 23° C. preferably represents from 9 to 97% by weight of the compound. This liquid fraction at 23° C. preferably represents between 15 and 85% by weight, and more preferably between 40 and 85% by weight. The liquid fraction by weight of the pasty compound at 23° C. is equal to the ratio of the enthalpy of fusion consumed at 23° C. to the enthalpy of fusion of the pasty compound.

The enthalpy of fusion of the pasty compound is the enthalpy consumed by the compound to change from the solid state to the liquid state. The pasty compound is said to be in the solid state when the whole of its mass is in crystalline solid form. The pasty compound is said to be in the liquid state when the whole of its mass is in the liquid form.

The enthalpy of fusion of the pasty compound is equal to the area under the curve of the thermogram obtained using a differential scanning calorimeter (DSC), such as the calorimeter sold under the name MDSC 2920 by TA Instrument, with a rise in temperature of 5 or 10° C. per minute, according to standard ISO 11357-3:1999. The enthalpy of fusion of the pasty compound is the amount of energy necessary to change the compound from the solid state to the liquid state. It is expressed in J/g.

The enthalpy of fusion consumed at 23° C. is the amount of energy absorbed by the sample to change from the solid state to the state which it exhibits at 23° C., constituted of a liquid fraction and of a solid fraction.

The liquid fraction of the pasty compound measured at 32° C. preferably represents from 30 to 100% by weight of the compound, preferably from 80 to 100%, more preferably from 90 to 100% by weight of the compound. When the liquid fraction of the pasty compound measured at 32° C. is equal to 100%, the temperature of the end of the melting range of the pasty compound is less than or equal to 32° C.

The liquid fraction of the pasty compound measured at 32° C. is equal to the ratio of the enthalpy of fusion consumed at 32° C. to the enthalpy of fusion of the pasty compound. The enthalpy of fusion consumed at 32° C. is calculated in the same way as the enthalpy of fusion consumed at 23° C.

The pasty compounds are generally hydrocarbon-based compounds such as lanolins and their derivatives or else PDMS compounds.

In particular, the pasty compound is preferably chosen from synthetic compounds and compounds of plant origin. A pasty compound may be obtained by synthesis from starting products of plant origin.

The presence of a pasty compound may make it possible to advantageously confer improved comfort during deposition of a composition of the invention on the keratin fibres.

Such a compound may advantageously be chosen from:
lanolin and its derivatives;
polymeric or non-polymeric silicone compounds;
polymeric or non-polymeric fluorinated compounds;
vinyl polymers, especially:
   olefin homopolymers;
   olefin copolymers;
   hydrogenated diene homopolymers and copolymers;
   linear or branched oligomers which are homopolymers or copolymers of alkyl (meth)acrylates preferably having a C₈-C₃₀ alkyl group;
   oligomers which are homopolymers and copolymers of vinyl esters having C₈-C₃₀ alkyl groups;
   oligomers which are homopolymers and copolymers of vinyl ethers having C₈-C₃₀ alkyl groups;
liposoluble polyethers resulting from the polyetherification between one or more C₂-C₁₀₀, preferably C₂-C₅₀, diols;
fatty acid or alcohol esters;
and mixtures thereof.

Among the esters, mention may especially be made of:
esters of a glycerol oligomer, especially diglycerol esters, in particular condensates of adipic acid and of glycerol, for which some of the hydroxyl groups of the glycerols have reacted with a mixture of fatty acids such as stearic acid, capric acid, stearic and isostearic acid and 12-hydroxystearic acid, just like, in particular, those sold under the brand name SOFTISAN 649 by Sasol or such as bis-diglyceryl polyacyladipate-2;
arachidyl propionate sold under trade name WAXENOL 801 by Alzo;
phytosterol esters,
fatty acid triglycerides and derivatives thereof, such as hydrogenated coco-glycerides;
non-crosslinked polyesters resulting from poly-condensation between a linear or branched C₄-C₅₀ dicarboxylic acid or polycarboxylic acid and a C₂-C₅₀ diol or polyol;
aliphatic esters of an ester resulting from the esterification of an aliphatic hydroxycarboxylic acid ester with an aliphatic carboxylic acid (SALACOS HCIS (V)-L sold by Nishing Oil);
polyesters resulting from the esterification, with a polycarboxylic acid, of an aliphatic hydroxy-carboxylic acid ester, said ester comprising at least two hydroxyl groups, such as the products RISOCAST DA-H® and RISOCAST DA-L®;
and mixtures thereof.

The pasty compound may also be chosen from compounds of plant origin. Among these compounds of plant origin, mention may especially be made of orange wax, such as for example the product sold under the reference ORANGE PEEL WAX by Koster Keunen, shea butter, partially hydrogenated olive oil such as for example the compound sold under the reference BEURROLIVE by Soliance, or else cocoa butter.

The pasty compounds may be used in an amount ranging from 1 to 50% by weight, in particular from 3 to 45% and more particularly from 5 to 40% by weight relative to the total weight of the composition.

Semi-Crystalline Polymers

The expression “semi-crystalline polymer” is understood, for the purposes of the invention, to mean polymers comprising a crystallizable portion, which is a pendent chain or a block in the backbone, and an amorphous portion in the backbone, and having a first-order reversible phase-change temperature, in particular of melting (solid-liquid transition). When the crystallizable portion is a block of the polymer backbone, this crystallizable block is of different chemical nature to that of the amorphous blocks; in this case, the semi-crystalline polymer is a block polymer, for example, of the diblock, triblock or multiblock type.

Advantageously, the semi-crystalline polymer(s) of the composition of the invention has (have) a number-average molecular weight M̅ₙ greater than or equal to 2000, ranging for example from 2000 to 800 000, preferably from 3000 to 500 000, for example from 4000 to 150 000 and better still from 4000 to 99 000.

In the composition according to the invention the semi-crystalline polymers are advantageously soluble in the oily phase to at least 1% by weight, at a temperature above their melting temperature. Apart from the crystallizable chains or blocks, the blocks of the polymers are amorphous. The expression “crystallizable chain or block” is understood, for the purposes of the invention, to mean a chain or block which, if it were alone, would change from the amorphous state to the crystalline state reversibly, depending on whether one is above or below the melting temperature. For the purposes of the invention, a “chain” is a group of atoms, which are pendent or lateral relative to the polymer backbone. A “block” is a group of atoms belonging to the backbone, this group constituting one of the repeating units of the polymer.

Preferably, the polymer backbone of the semi-crystalline polymers is soluble in the oily phase.

Preferably, the semi-crystalline polymers used in the composition of the invention have a melting temperature (or melting point), pF, below 70° C. (25° C.≦pF<70° C.), this temperature being at least equal to the temperature of the keratin material that has to receive the composition according to the invention, in particular the skin. The melting temperature may be measured by any known method and in particular using a differential scanning calorimeter (DSC).

Preferably, the crystallizable blocks or chains of the semi-crystalline polymers represent at least 30% of the total weight of each polymer and better still at least 40%. The semi-crystalline polymers containing crystallizable blocks used according to the invention are block or multiblock polymers. They may be obtained by polymerizing monomers containing reactive (or ethylenic) double bonds or by polycondensation. When the polymers of the invention are polymers containing crystallizable side chains, these side chains are advantageously in random or statistical form.

The semi-crystalline polymers of the invention are of synthetic origin. Moreover, they do not comprise a polysaccharide backbone.

The semi-crystalline polymers that can be used in the invention are preferably chosen from polymers (homopolymers and copolymers) bearing at least one crystallizable side chain, and polymers (homopolymers and copolymers) bearing, in the backbone, at least one crystallizable block, for instance those described in document U.S. Pat. No. 5,156,911. The crystallizable side chains or blocks are hydrophobic.

According to one preferred embodiment of the invention, the semi-crystalline polymers are especially chosen from the homopolymers and copolymers resulting from the polymerization of at least one monomer containing crystallizable chain(s), the latter being chosen from the alkyl chains comprising at least 11 carbon atoms and at most 40 carbon atoms, and better still at most 24 carbon atoms. These are alkyl chains comprising at least 12 carbon atoms, and preferably these are alkyl chains comprising from 14 to 24 carbon atoms (C₁₄-C₂₄). They may be hydrocarbon-based alkyl chains (carbon and hydrogen atoms) or fluorinated or perfluorinated alkyl chains (carbon atoms, fluorine atoms and possibly hydrogen atoms). When they are fluorinated or perfluorinated alkyl chains, they comprise at least 11 carbon atoms, at least 6 carbon atoms of which are fluorinated.

The term “alkyl” is understood to mean a saturated group (that does not comprise an unsaturation).

According to one particular embodiment of the invention, the semi-crystalline polymer is chosen from the homopolymers obtained by polymerization of at least one monomer containing a crystallizable chain, chosen from C₁₄-C₂₄ alkyl (meth)acrylates, perfluoro(C₁₁-C₁₅)alkyl (meth)acrylates, N—(C₁₄ to C₂₄)alkyl (meth)acrylamides with or without a fluorine atom, vinyl esters containing C₁₄-C₂₄ alkyl or perfluoro(C₁₄-C₂₄)alkyl chains, vinylene ethers containing C₁₄-C₂₄ alkyl or perfluoro(C₁₄-C₂₄)alkyl chains, C₁₄-C₂₄ alpha-olefins, para-alkylstyrenes with a C₁₄-C₂₄ alkyl group, and from the copolymers of these monomers, obtained by copolymerization of these monomers with a hydrophilic monomer, preferably other than methacrylic acid, such as for example N-vinylpyrrolidone, hydroxyethylacrate, hydroxyethylmethacrylate, acrylic acid. Such copolymers may, for example, be copolymers of C₁₄-C₂₄ alkyl acrylate, C₁₄-C₂₄ alkyl methacrylate, C₁₄-C₂₄ alkyl acrylamide, C₁₄-C₂₄ alkyl methacrylamide with N-vinylpyrrolidone, hydroxyethylacrylate, hydroxyethyl-methacrylate, acrylic acid, or mixtures thereof.

Preferably, the semi-crystalline polymer is chosen from the homopolymers obtained by polymerization of a monomer chosen from C₁₄-C₂₄ alkyl acrylates and C₁₄-C₂₄ alkyl methacrylates and from the copolymers obtained by copolymerization of a monomer chosen from C₁₄-C₂₄ alkyl acrylates and C₁₄-C₂₄ alkyl methacrylates, with a hydrophilic monomer such as acrylic acid.

The semi-crystalline polymers of the composition of the invention may be uncrosslinked or partly crosslinked, as long as the degree of crosslinking does not hinder their dissolution or dispersion in the oily phase by heating above their melting temperature. This may then be a chemical crosslinking, by reaction with a multifunctional monomer during the polymerization. This may also be a physical crosslinking which may then be due either to establishing hydrogen or dipolar type bonds between groups borne by the polymer, such as for example dipolar interactions between carboxylate ionomers, these interactions being in a small amount and borne by the backbone of the polymer; or to a phase separation between the crystallizable blocks and the amorphous blocks borne by the polymer.

Preferably, the semi-crystalline polymers of the composition according to the invention are not crosslinked.

According to one particular embodiment of the invention, the semi-crystalline polymer is a homopolymer resulting from the polymerization of a monomer containing a crystallizable chain chosen from C₁₄-C₂₄ alkyl acrylates and C₁₄-C₂₄ alkyl methacrylates. Mention may especially be made of those sold under the names INTELIMER® by Landec, described in the brochure “Intelimer® polymers”, Landec IP22. These polymers are in solid form at room temperature. They bear crystallizable side chains and correspond to homopolymers of saturated C₁₄-C₂₄ alkyl acrylates or methacrylates. Mention may more particularly be made of the stearyl acrylate homopolymer (INTELIMER IPA-13.1) (INCI name: Poly C10-30 alkyl acrylate), the behenyl acrylate homopolymer (INTELIMER IPA-13.6) (INCI name: Poly C10-30 alkyl acrylate).

According to another particular embodiment of the invention, the semi-crystalline polymer is a copolymer of C₁₄-C₂₄ alkyl acrylates or of C₁₄-C₂₄ alkyl methacrylates with acrylic acid. As copolymers of this type, mention may be made of the copolymers obtained by the copolymerization of behenyl acrylate and of acrylic acid, and the copolymers obtained by the copolymerization of stearyl acrylate and acrylic acid.

According to one preferred embodiment of the invention, the semi-crystalline polymer is a homopolymer, and it is chosen from the stearyl acrylate homopolymer (INTELIMER IPA-13.1) (INCI name: Poly C10-30 alkyl acrylate), the behenyl acrylate homopolymer (INTELIMER IPA-13.6) (INCI name: Poly C10-30 alkyl acrylate), and mixtures thereof.

The semi-crystalline polymers may be used in an amount ranging from 1 to 50% by weight, in particular from 3 to 45%, and more particularly from 5 to 40% by weight relative to the total weight of the composition.

Lipophilic Gelling Agents

The gelling agents that can be used in the compositions according to the invention may be organic or mineral, polymeric or molecular lipophilic gelling agents.

Mineral lipophilic gelling agents that may be mentioned include optionally modified clays, for instance hectorites modified with a C₁₀ to C₂₂ fatty acid ammonium chloride, for instance hectorite modified with distearyldimethylammonium chloride, such as for example the product sold under the name BENTONE 38V® by Elementis.

Mention may also be made of fumed silica optionally subjected to a hydrophobic surface treatment, the particle size of which is less than 1 μm. Specifically, it is possible to chemically modify the surface of the silica, by chemical reaction generating a reduced number of silanol groups present at the surface of the silica. It is especially possible to substitute silanol groups with hydrophobic groups: a hydrophobic silica is then obtained. The hydrophobic groups may be:
trimethylsiloxyl groups, which are obtained especially by treating fumed silica in the presence of hexamethyldisilazane. Silicas thus treated are known as “silica silylate” according to the CTFA (6th edition, 1995). They are sold, for example, under the references AEROSIL R812® by Degussa, and CAB-O-SIL TS-530® by Cabot;
dimethylsilyloxyl or polydimethylsiloxane groups, which are obtained especially by treating fumed silica in the presence of polydimethylsiloxane or dimethyldichlorosilane. Silicas thus treated are known as “silica dimethyl silylate” according to the CTFA (6th edition, 1995). They are sold, for example, under the references AEROSIL R972® and AEROSIL R974® by Degussa, and CAB-O-SIL TS-610® and CAB-O-SIL TS-720® by Cabot.

The hydrophobic fumed silica in particular has a particle size that may be nanometric to micrometric, for example ranging from about 5 to 200 nm.

The polymeric organic lipophilic gelling agents are, for example, partially or completely crosslinked elastomeric organopolysiloxanes of three-dimensional structure, for instance those sold under the names KSG6®, KSG16® and KSG18® by Shin-Etsu, TREFIL E-505C® and TREFIL E-506C® by Dow Corning, GRANSIL SR-CYC®, SR DMF10®, SR-DC556®, SR 5CYC GEL®, SR DMF 10 GEL® and SR DC 556 GEL® by Grant Industries and SF 1204® and JK 113® by General Electric; ethyl cellulose, for instance the product sold under the name ETHOCEL® by Dow Chemical; polycondensates of polyamide type resulting from the condensation between (α) at least one acid chosen from dicarboxylic acids containing at least 32 carbon atoms, such as fatty acid dimers, and (β) an alkylenediamine and in particular ethylenediamine, in which the polyamide polymer comprises at least one carboxylic acid end group esterified or amidated with at least one saturated and linear monoalcohol or one saturated and linear monoamine containing from 12 to 30 carbon atoms, and in particular ethylenediamine/stearyl dilinoleate copolymers such as the product sold under the name UNICLEAR 100 VG® by Arizona Chemical; silicone polyamides of the polyorganosiloxane type, for instance those described in documents U.S. Pat. No. 5,874,069, U.S. Pat. No. 5,919,441, U.S. Pat. No. 6,051,216 and U.S. Pat. No. 5,981,680, such as for example those sold under the reference DOW CORNING 2-8179 GELLANT and DOW CORNING 2-8178 GELLANT by Dow Corning; galactomannans comprising from one to six and in particular from two to four hydroxyl groups per saccharide, substituted with a saturated or unsaturated alkyl chain, for instance guar gum alkylated with C₁ to C₆, and in particular C₁ to C₃, alkyl chains, and mixtures thereof. Block copolymers of “diblock”, “triblock” or “radial” type, of the polystyrene/polyisoprene or polystyrene/polybutadiene type, such as the products sold under the name LUVITOL HSB® by BASF, of the polystyrene/copoly(ethylene-propylene) type, such as the products sold under the name KRATON® by Shell Chemical Co., or of the polystyrene/copoly(ethylene-butylene) type, and mixtures of triblock and radial (star) copolymers in isododecane, such as those sold by Penreco under the name VERSAGEL®, for instance the mixture of butylene/ethylene/styrene triblock copolymer and of ethylene/propylene/styrene star copolymer in isododecane (VERSAGEL M 5960).

The compositions according to the invention may also comprise, as a lipophilic gelling agent, a non-emulsifying silicone elastomer. Non-emulsifying elastomers are especially described in patent applications JP-A-61-194 009, EP-A-242 219, EP-A-285 886 and EP-A-765 656. As spherical non-emulsifying elastomers, use may be made of those sold under the names DC 9040, DC9041, DC 9509, DC9505 and DC 9506 by Dow Corning. The spherical non-emulsifying silicone elastomer may also be in the form of elastomeric crosslinked organopolysiloxane powder coated with silicone resin, especially with silsesquioxane resin, as described, for example, in U.S. Pat. No. 5,538,793. Such elastomers are sold under the names KSP-100, KSP-101, KSP-102, KSP-103, KSP-104, KSP-105 by Shin Etsu.

Other elastomeric crosslinked organopolysiloxanes in the form of spherical powders may be hybrid silicone powders functionalized with fluoroalkyl groups, sold especially under the name KSP-200 by Shin-Etsu; hybrid silicone powders functionalized with phenyl groups, sold especially under the name KSP-300 by Shin-Etsu.

Use may also be made, in the compositions according to the invention, of silicone elastomers with an MQ group, such as those sold by Wacker under the names BELSIL RG100, BELSIL RPG33 and preferentially RG80. These particular elastomers, when they are in combination with the resins according to the invention, may make it possible to improve the non-transfer properties of the compositions comprising them.

Among the lipophilic gelling agents, mention may also be made of organogelling agents and in particular:
bis-urea derivatives of general formula (I):

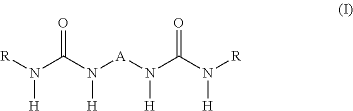

in which:
A is a group of formula:

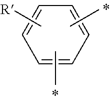

with R′ being a linear or branched C₁ to C₄ alkyl radical, and the *s symbolizing the points of attachment of the group A to each of the two nitrogen atoms of the rest of the compound of general formula (I), and
R is a saturated or unsaturated, non-cyclic, monobranched C₆ to C₁₅ alkyl radical, the hydrocarbon-based chain of which is optionally interrupted by 1 to 3 heteroatoms chosen from O, S and N, or

a salt or isomer thereof in particular described in patent application FR-A-2892303;
silicone bis-urea derivatives of general formula (I) or a salt and/or isomer thereof:

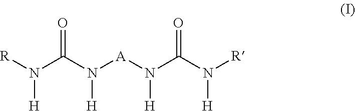

in which:
A is a group of formula (II):

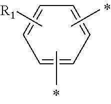

with R₁ being a linear or branched C₁ to C₄ alkyl radical, and the *s symbolizing the points of attachment of the group A to each of the two nitrogen atoms of the rest of the compound of general formula (I), and
R and R′, which may be identical or different, are chosen from:
i) the radicals of formula (III):

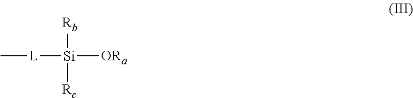

in which:
L is a single bond or a divalent carbon-based radical, especially a linear, branched and/or cyclic, saturated or unsaturated hydrocarbon-based radical (alkylene), comprising 1 to 18 carbon atoms, and possibly comprising 1 to 4 heteroatoms chosen from N, O and S;
Rₐ is:

a) a carbon-based radical, especially a linear, branched and/or cyclic, saturated or unsaturated hydrocarbon-based radical (alkyl), comprising 1 to 18 carbon atoms, and possibly comprising 1 to 8 heteroatoms chosen from N, O, Si and S; or

b) a silicone radical of formula:

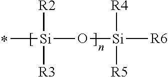

with n being between 0 and 100, especially between 1 and 80, or even 2 to 20;

and R₂ to R₆ being, independently of each other, carbon-based radicals, especially linear or branched hydrocarbon-based radicals (alkyl) comprising 1 to 12 and especially 1 to 6 carbon atoms, and possibly comprising 1 to 4 heteroatoms, especially O;
R_{b} and R_{c} are, independently of each other, chosen from:

a) carbon-based radicals, especially linear, branched and/or cyclic, saturated or unsaturated hydrocarbon-based radicals (alkyl), comprising 1 to 18 carbon atoms, and possibly comprising 1 to 4 heteroatoms chosen from N, O, Si and S;

b) the radicals of formula:

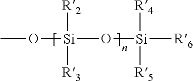

with n being between 0 and 100, especially between 1 and 80, or even 2 to 20;

and R′₂ to R′₆ being, independently of each other, carbon-based radicals, especially linear or branched hydrocarbon-based radicals (alkyl), comprising 1 to 12 and especially 1 to 6 carbon atoms, and possibly comprising 1 to 4 heteroatoms, especially O; and
ii) linear, branched and/or cyclic, saturated or unsaturated C₁ to C₃₀ alkyl radicals, optionally comprising 1 to 3 heteroatoms chosen from O, S, F and N;

it being understood that at least one of the radicals R and/or R′ is of formula (III) such as those described in patent application FR-A-2900819.
The bis-urea derivatives described in patent application FR-A-2894476.

Among the other lipophilic gelling agents that may be used in the compositions according to the invention, mention may also be made of fatty alcohols comprising from 10 to 30 carbon atoms. The expression “fatty alcohols comprising from 10 to 30 carbon atoms” is understood to mean any branched or unbranched, saturated or unsaturated, pure fatty alcohol comprising from 10 to 30 carbon atoms.

Use is preferably made of a fatty alcohol comprising from 10 to 26 carbon atoms, better still from 10 to 24 carbon atoms and even better still from 14 to 22 carbon atoms.

As fatty alcohols that can be used, mention may especially be made of lauryl, myristyl, cetyl, stearyl, oleyl, cetearyl (mixture of cetyl alcohol and stearyl alcohol), behenyl and erucyl alcohols and mixtures thereof. Preferably, cetyl alcohol or behenyl alcohol is used.

Such fatty alcohols are especially sold under the name NAFOL by Sasol.

Among the lipophilic gelling agents that may be used in the compositions according to the invention, mention may also be made of fatty acid esters of dextrin, such as dextrin palmitates, especially the products sold under the name RHEOPEARL TL® or RHEOPEARL KL® by Chiba Flour.

The lipophilic gelling agents may be used in an amount ranging from 1 to 60% by weight, in particular from 2 to 50%, and more particularly from 5 to 40% by weight relative to the total weight of the composition.

Additives

Dyestuff

According to the invention or according to one advantageous embodiment of the invention, according to the subject of the invention in question, the makeup compositions comprise at least one dyestuff chosen from pulverulent dyestuffs.

The pulverulent dyestuffs may be chosen from pigments and pearlescent agents.

The pigments may be white or coloured, mineral and/or organic, and coated or uncoated. Among the mineral pigments which may be mentioned are titanium dioxide, optionally surface-treated, zirconium oxide, zinc oxide or cerium oxide, and also iron oxide, chromium oxide, manganese violet, ultramarine blue, chromium hydrate and ferric blue. Among the organic pigments that may be mentioned are carbon black, pigments of D & C type, and lakes based on cochineal carmine or on barium, strontium, calcium or aluminium.

The pearlescent agents include iridescent or noniridescent coloured particles of any shape, which are produced in particular by certain molluscs in their shell or else are synthesized, and which exhibit a colour effect by optical interference. As examples of pearlescent agents, mention may be made of pearlescent pigments, such as titanium mica coated with an iron oxide, mica coated with bismuth oxychloride, titanium mica coated with chromium oxide and pearlescent pigments based on bismuth oxychloride. This may also involve mica particles, at the surface of which at least two successive layers of metal oxides and/or of organic dyestuffs are superposed. The pearlescent agents may more particularly have a yellow, pink, red, bronze, orangey, brown, gold and/or coppery colour or tint.

The compositions according to the invention may also comprise, in addition, at least one dyestuff chosen from liposoluble dyes and water-soluble dyes.

The liposoluble dyes are, for example, Sudan Red, D&C Red 17, D&C Green 6, β-carotene, soybean oil, Sudan Brown, D&C Yellow 11, D&C Violet 2, D&C Orange 5, quinoline yellow and annatto.

The pulverulent materials may be present in a content ranging from 1 to 15% by weight relative to the total weight of the composition, preferably from 5 to 10% by weight.

Generally, these dyestuffs may be present in a content ranging from 0.01% to 30% by weight relative to the total weight of the composition.

According to one particular embodiment, the iron oxides will be present in a content ranging from 0.01% to 15% by weight, preferably 0.01% to 10% by weight, relative to the total weight of the composition.

Fibres

The compositions according to the invention may also comprise fibres which enable an improvement in the lengthening effect.

The term “fibre” should be understood as meaning an object of length L and diameter D such that L is very much greater than D, D being the diameter of the circle in which the cross section of the fibre is inscribed. In particular, the ratio L/D (or shape factor) is chosen in the range from 3.5 to 2500, in particular from 5 to 500 and more particularly from 5 to 150.

The fibres that may be used in the composition of the invention may be mineral or organic fibres of synthetic or natural origin. They may be short or long, individual or organized, for example braided, and hollow or solid. They may have any shape, and may especially have a circular or polygonal (square, hexagonal or octagonal) cross section, depending on the intended specific application. In particular, their ends are blunt and/or polished to prevent injury.

In particular, the fibres have a length ranging from 1 μm to 10 mm, in particular from 0.1 mm to 5 mm and more particularly from 0.3 mm to 3.5 mm. Their cross section may be within a circle of diameter ranging from 2 nm to 500 μm, in particular ranging from 100 nm to 100 μm and more particularly from 1 μm to 50 μm. The weight or yarn count of the fibres is often given in denier or decitex, and represents the weight in grams per 9 km of yarn. In particular, the fibres according to the invention may have a yarn count chosen in the range from 0.15 to 30 denier and especially from 0.18 to 18 denier.

The fibres that may be used in the composition of the invention may be chosen from rigid or non-rigid fibres, and may be mineral or organic, of synthetic or natural origin.

Moreover, the fibres may or may not be surface-treated, may be coated or uncoated, and may be coloured or uncoloured.

As fibres that may be used in the composition according to the invention, mention may be made of non-rigid fibres such as polyamide (Nylon®) fibres or rigid fibres such as polyimideamide fibres, for instance those sold under the names KERMEL® and KERMEL TECH® by Rhodia or poly(p-phenyleneterephthalamide) (or aramid) fibres sold especially under the name KEVLAR® by DuPont de Nemours.

The fibres may be present in the composition according to the invention in a content ranging from 0.01% to 10% by weight, in particular from 0.1% to 5% by weight and more particularly from 0.3% to 3% by weight relative to the total weight of the composition.

Cosmetic Active Agents

As cosmetic active agents that can be used in the compositions according to the invention, mention may especially be made of antioxidants, preservatives, fragrances, neutralizers, emollients, moisturizers, vitamins and screening agents, in particular sunscreens.

Of course, a person skilled in the art will be sure to choose the optional additional additives and/or the amount thereof so that the advantageous properties of the composition according to the invention are not, or are not substantially, impaired by the intended addition.

Makeup-Removing and/or Cleansing Composition

The makeup-removing and/or cleansing composition may comprise predominantly water and/or a water-soluble solvent, for example in a content greater than or equal to 50%, better still greater than or equal to 80% by weight relative to the total weight of said composition.

In order to achieve this makeup removal, at least one finger may be impregnated with this composition. As a variant, a support may be intended to be impregnated and thus be dry or anhydrous. As a variant, this support may be pre-impregnated with the composition.

This support may be a woven or non-woven fabric, which is optionally laminated, such as a Demakeup® disc.

Making-Up and Makeup-Removing and/or Cleansing Kit

This kit may comprise a makeup composition according to the invention in combination with a makeup-removing and/or cleansing composition.

This makeup-removing and/or cleansing composition may be packaged with said makeup composition, separately, in one and the same packaging article.

The makeup composition may be a makeup product for the lips, such as a lipstick, a lip balm, a lip gloss or a lip pencil, a complexion product, such as a foundation, a loose or pressed powder, a face powder or eye shadow, an anti-wrinkle product, a blusher, a mascara, an eyeliner or else a product for making up the body or for colouring the skin.

The makeup-removing and/or cleansing composition may be as described previously.

### Examples Relating to the Use of a Particular Emulsifying System

Mascara formulations are prepared using deionized water as follows.

### Example 1

### Composition Outside of the Invention

| Ingredients | % |
|---|---|
| water | 45.5 |
| Hydroxyethyl cellulose (HEC) | 0.7 |
| Triethanolamine (TEA) | 2 |
| glyceryl stearate | 2.5 |
| Beeswax | 10 |
| Stearic acid | 5 |
| preservatives | 0.3 |
| black iron oxide | 10 |
| Simethicone | 0.5 |
| Dispersion of polyurethane in water | 23.5 |
| containing 43% of active material | |

The beeswax is melted at 95° C. with the preservatives and the stearic acid. Next the black iron oxide is dispersed using a Moritz device. Next, the hydroxyethyl cellulose (HEC) then the triethanolamine and the glyceryl stearate are dispersed, in water brought to 95° C., using a Rayneri device. Using a Moritz stirrer, the aqueous phase is added to the fatty phase until the emulsion is formed. This emulsion is cooled while gently stirring. At around 30° C. the polyurethane is slowly added.

Products used in this example:
Glyceryl stearate sold under the name TEGIN M PELLETS by Evonic Goldschmidt GmbH, Essen,
Simethicone sold under the name MIRASIL SM by Rhodia Silicones S.A.S., Lyon,
Polyurethane dispersed in water is sold under the name BAYCUSAN C 1000 by Bayer MaterialScience LLC, Pittsburgh, Pa.,

the other ingredients being readily available to a person skilled in the art.

The result of the makeup-removal test is the absence of sheaths.

### Example 2

### Composition 1 According to the Invention

| Ingredients | % |
|---|---|
| water | 36.48 |
| Hydroxyethyl cellulose (HEC) | 0.7 |
| Beeswax | 10 |
| Cetyl alcohol | 2 |
| Stearyl alcohol 20(EO) | 4.44 |
| Stearyl alcohol 2(EO) | 2.1 |
| Potassium cetyl phosphate | 2.18 |
| Preservative | 0.3 |
| Black iron oxide | 10 |
| Simethicone | 0.5 |
| Dispersion of polyurethane in water | 31.3 |
| containing 32% of active material | |

This composition is prepared in the same way as described previously, only the emulsifying system changing, the surfactant system being replaced by an emulsifying system comprising cetyl alcohol, 20 EO stearyl alcohol, 2 EO stearyl alcohol and potassium cetyl phosphate.

Products used in this example:
Cetyl alcohol sold under the name PHYTOWAX ricin 16L64 and 22L73 by SOPHIM,
Stearyl alcohol 20(EO) sold under the name BRIJ 78 by UNIQEMA,
Stearyl alcohol 2(EO) sold under the name BRIJ 72 by UNIQEMA,
Potassium cetyl phosphate sold under the name AMPHISOL K by Givaudan,
Simethicone sold under the name MIRASIL SM by Rhodia Silicones S.A.S., Lyon,
Polyurethane dispersed in water is sold under the name BAYCUSAN C 1001 by Bayer MaterialScience LLC, Pittsburgh, Pa.,

the other ingredients being readily available to a person skilled in the art.

The result of the makeup-removal test with such a composition according to the protocol described above is a makeup-removal via sheaths.

### Example 3

### Composition 2 According to the Invention

| Ingredients | % |
|---|---|
| water | 41.7 |
| Paraben | 0.25 |
| Phenoxyethanol and parabens | 1.2 |
| Hydroxybenzoate | 0.15 |
| EDTA | 0.2 |
| Sodium dehydroacetate | 0.2 |
| PEG 200 glyceryl stearate | 4 |
| 1,3-Butylene glycol | 5 |
| Black pigment | 7 |
| Acrylamide copolymer | 2.5 |
| Beeswax | 7.4 |
| Carnauba wax | 3.5 |
| Paraben | 0.05 |
| Simethicone | 0.1 |
| Dispersion of polyurethane in water | 23.75 |
| containing 32% of active material | |
| ethanol | 3 |

This formulation is prepared as follows:
Heat the water to 95° C. and disperse, using the Rayneri device, the preservatives, the surfactant, the butylene glycol then the pigments. Add the acrylamide copolymer.
Melt the fatty phase at 95° C. Add to the aqueous phase using the Rayneri stirrer, stir for 10 minutes then leave to cool while gently stirring. At around 30° C. slowly add the polyurethane then the ethanol.

Products used in this example:
PEG 200 glyceryl stearate sold under the name SIMULSOL 220 by SEPIC S.A., Paris,
1,3-Butylene glycol sold, for example, by Celanese Chemical, Dallas,
Acrylamide copolymer sold under the name SIMULGEL 600 by SEPPIC S.A., Paris,
Simethicone sold under the name MIRASIL SM by Rhodia Silicones,
Polyurethane dispersed in water is sold under the name BAYCUSAN C 1001 by Bayer MaterialScience LLC, Pittsburgh, Pa.,

the other ingredients being readily available to a person skilled in the art.

The result of the makeup-removal test with such a composition is a makeup-removal via sheaths.

### Examples Relating to the Use of a Particular Hydrophilic Thickener

Protocols:

In order to evaluate the in vitro “water resistance” of a composition, the latter is evaluated according to the following protocol:
The composition is applied to 3 samples of straight Caucasian hair of 30 knots (60 eyelashes having a length of 1 cm), fringe length of 2 cm, by making 3×10 passes at 2 minute intervals with uptake of product between each series of 10 passes. Each sample is then dried at room temperature for a drying time of one hour.

The 3 made-up samples are immersed in a vessel containing water at 20° C. for a given time (1 hour, 24 hours or 1 week). The 3 samples are then wiped back and forth 5 times on a square cloth of the Wypall L40 type from Kimberly-Clark.
The presence of marks deposited by the sample is then evaluated.
A score between 0 and 9 is given to the resulting marks; 0 being the score obtained when no mark is deposited by the sample, and 9 the score obtained when very large marks are deposited.

The expression “sebum resistance” is understood according to the present application to mean the in vitro sebum resistance evaluated according to the same measurement protocol as for the water resistance described above, except that the 3 made-up samples are immersed in a vessel containing squalene (squalene is present at a concentration of 18% in the composition of sebum), instead of water.

The expression “resistance to rubbing” is understood according to the present application to mean the in vitro resistance to rubbing evaluated according to the following protocol:
The composition is applied to 3 samples of straight Caucasian hair of 30 knots (60 eyelashes having a length of 1 cm), fringe length of 2 cm, by making 3×10 passes at 2 minute intervals with uptake of product between each series of 10 passes. Each sample is then dried at room temperature for a drying time of one hour.
The made-up sample is then positioned perpendicularly above a sheet of paper and rubbed using a hard brush of Keracils° type (30 passes). The quantity of grains thus formed, recovered on the sheet of paper, is evaluated.
A score between 0 and 6 is given to this quantity of grains; 0 being the score for which no grains are recovered on the sheet of paper, and 6 the score for which a very large quantity of grains is recovered.

Mascara formulations are prepared using deionized water as follows.

### Example 1

### Comparative Composition

| Ingredients | % |
|---|---|
| Water | 42.7 |
| Paraben | 0.25 |
| Phenoxyethanol and parabens mixture | 1.2 |
| Hydroxybenzoate | 0.15 |
| EDTA | 0.2 |
| Sodium dehydroacetate | 0.2 |
| Hydroxyethyl cellulose (HEC) | 1 |
| PEG 200 Glyceryl stearate | 4 |
| 1,3-Butylene glycol | 5 |
| Black pigment | 7 |
| Polysorbate 80 | 0.5 |
| Beeswax | 7.4 |
| Carnauba | 3.5 |
| Paraben | 0.05 |
| Simethicone | 0.1 |
| Latex | 23.75 |
| Ethanol | 3 |

This formulation is prepared as follows:
Heat the water to 95° C. and disperse, using the Rayneri device, the preservatives, the surfactant and the HEC, the butylene glycol then the pigments. Add the acrylamide copolymer.
Melt the fatty phase at 95° C. Add to the aqueous phase using the Rayneri stirrer, stir for 10 minutes then leave to cool while gently stirring. At around 30° C. slowly add the polyurethane then the ethanol.

Products used in this example:
PEG 200 glyceryl stearate sold under the name SIMULSOL 220 by SEPPIC S.A., Paris,
1,3-Butylene glycol sold, for example, by Celanese Chemical, Dallas,
Simethicone sold under the name MIRASIL SM by Rhodia Silicones,
Polyurethane dispersed in water is sold under the name BAYCUSAN C 1001 by Bayer MaterialScience LLC, Pittsburgh, Pa.,

the other ingredients being readily available to a person skilled in the art.

The protocols for evaluating the resistance described previously gave the following results with this composition:

| Water | Water | Sebum | |
|---|---|---|---|
| resistance | resistance | resistance | Dry |
| 1 min | 1 h | 1 min | strength |
| 2 | 3 | 1 | 5 |

### Example 2

### Composition with Hydrophilic Thickener

| Ingredients | % |
|---|---|
| Water | 43.4 |
| Paraben | 0.25 |
| Phenoxyethanol and parabens | 1.2 |
| mixture | |
| Hydroxybenzoate | 0.15 |
| EDTA | 0.2 |
| Sodium dehydroacetate | 0.2 |
| PEG 200 Glyceryl stearate | 4 |
| 1,3-Butylene glycol | 5 |
| Black pigment | 7 |
| Hydrophilic thickener | 0.8 |
| Beeswax | 7.4 |
| Carnauba | 3.5 |
| Paraben | 0.05 |
| Simethicone | 0.1 |
| Latex | 23.75 |
| Ethanol | 3 |

Heat the water to 95° C. and disperse, using the Rayneri device, the preservatives, the surfactant, the butylene glycol then the pigments. Add the acrylamide copolymer.
Melt the fatty phase at 95° C. Add to the aqueous phase using the Rayneri stirrer, stir for 10 minutes then leave to cool while gently stirring. At around 30° C. slowly add the polyurethane then the ethanol.

Products used in this example:
PEG 200 glyceryl stearate sold under the name SIMULSOL 220 by SEPPIC S.A., Paris,
1,3-Butylene glycol sold, for example, by Celanese Chemical, Dallas,
hydrophilic thickener sold under the name PEMULEN TR2 by Lubrizol,
Simethicone sold under the name MIRASIL SM by Rhodia Silicones,
polyurethane dispersed in water is sold under the name BAYCUSAN C 1001 by Bayer MaterialScience LLC, Pittsburgh, Pa.,

the other ingredients being readily available to a person skilled in the art.

Results

The protocols for evaluating the resistance described previously gave the following results with this composition:

| Water | Water | Sebum | |
|---|---|---|---|
| resistance | resistance | resistance | Dry |
| 1 min | 1 h | 1 min | strength |
| 0 | 0 | 1 | 4 |

It is thus observed, by comparing the two compositions, that the composition according to the invention containing a hydrophilic thickener of PEMULEN type considerably improves the water resistance, which is excellent and long-lasting.

The composition according to the invention also retains an excellent sebum resistance.

This composition furthermore has a good resistance to rubbing, which is improved.

### Examples Relating to the Use of a Lipophilic Plasticizer

Protocols

The same protocols described previously are applied to the following examples with a view to evaluating the water resistance, the sebum resistance and the resistance to rubbing of the compositions tested.

Mascara formulations are prepared using deionized water as follows.

### Example 1

### Comparative Composition

| Ingredients | % |
|---|---|
| Water | 42.7 |
| Paraben | 0.25 |
| Phenoxyethanol and parabens mixture | 1.2 |
| Hydroxybenzoate | 0.15 |
| EDTA | 0.2 |
| Sodium dehydroacetate | 0.2 |
| HEC | 1 |
| PEG 200 Glyceryl stearate | 4 |
| 1,3-Butylene glycol | 5 |
| Black pigment | 7 |
| Polysorbate 80 | 0.5 |
| Beeswax | 7.4 |
| Carnauba | 3.5 |
| Paraben | 0.05 |
| Simethicone | 0.1 |
| Dispersion of polyurethane in water | 23.75 |
| containing 32% of active material | |
| Ethanol | 3 |

This formulation is prepared as follows:
Heat the water to 95° C. and disperse, using the Rayneri device, the preservatives, the hydroxyethyl cellulose (HEC), the surfactant, the butylene glycol then the pigments. Add the acrylamide copolymer.
Melt the fatty phase at 95° C. Add to the aqueous phase using the Rayneri stirrer, stir for 10 minutes then leave to cool while gently stirring. At around 30° C. slowly add the polyurethane then the ethanol.

Products used in this example:
PEG 200 glyceryl stearate sold under the name SIMULSOL 220 by SEPPIC S.A., Paris,
1,3-Butylene glycol sold, for example, by Celanese Chemical, Dallas,
Polysorbate 80 sold under the name TWEEN 80 by Uniquema,
Simethicone sold under the name MIRASIL SM by Rhodia Silicones,
Polyurethane dispersed in water is sold under the name BAYCUSAN C 1001 by Bayer MaterialScience LLC, Pittsburgh, Pa.,

the other ingredients being readily available to a person skilled in the art.

The protocols for evaluating the resistance described previously gave the following results with this composition:

| Water | Water | Sebum | |
|---|---|---|---|
| resistance | resistance | resistance | Dry |
| 1 min | 1 h | 1 min | strength |
| 2 | 3 | 1 | 5 |

It is thus observed, by comparing the two compositions, that the composition according to the invention containing a lipophilic plasticizer of PEMULEN type considerably improves the water resistance, which is excellent.

The composition according to the invention also retains an excellent sebum resistance and has a good resistance to rubbing.

### Example 2

### Composition with Lipophilic Plasticizer

| Ingredients | % |
|---|---|
| Water | 36.7 |
| Paraben | 0.25 |
| Phenoxyethanol and parabens | 1.2 |
| Hydroxybenzoate | 0.15 |
| EDTA | 0.2 |
| Sodium dehydroacetate | 0.2 |
| PEG 200 Glyceryl stearate | 4 |
| 1,3-Butylene glycol | 5 |
| Black pigment | 7 |
| Acrylamide copolymer | 2.5 |
| Beeswax | 7.4 |
| Carnauba wax | 3.5 |
| Paraben | 0.05 |
| Simethicone | 0.1 |
| Dispersion of polyurethane in water | 23.75 |
| containing 32% of active material | |
| Triethyl citrate | 5 |
| Ethanol | 3 |

This formulation is prepared as follows:

Heat the water to 95° C. and disperse, using the Rayneri device, the preservatives, the surfactant, the butylene glycol then the pigments. Add the acrylamide copolymer.
Melt the fatty phase at 95° C. Add to the aqueous phase using the Rayneri stirrer, stir for 10 minutes then leave to cool while gently stirring. At around 30° C. slowly add the polyurethane then the ethanol.

Products used in this example:
PEG 200 glyceryl stearate sold under the name SIMULSOL 220 by SEPPIC S.A., Paris,
1,3-Butylene glycol sold, for example, by Celanese Chemical, Dallas,
Acrylamide copolymer sold under the name SIMULGEL 600 by SEPPIC S.A., Paris,
Simethicone sold under the name MIRASIL SM by Rhodia Silicones,
Polyurethane dispersed in water is sold under the name BAYCUSAN C 1001 by Bayer MaterialScience LLC, Pittsburgh, Pa.,
Triethyl citrate sold under the name CITROFLEX° 2 by Morflex,

the other ingredients being readily available to a person skilled in the art.

Result

The protocols for evaluating the resistance described previously gave the following results with this composition:

| Water | Water | Sebum | |
|---|---|---|---|
| resistance | resistance | resistance | Dry |
| 1 min | 1 h | 1 min | strength |
| 1 | 2 | 1 | 2 |

It is thus observed, by comparing the two compositions, that the composition according to the invention containing a lipophilic plasticizer of triethyl citrate type considerably improves the water resistance, which is excellent.

The composition according to the invention also retains an excellent sebum resistance.

This composition furthermore has a very good resistance to rubbing, which is significantly improved.

The above written description of the invention provides a manner and process of making and using it such that any person skilled in this art is enabled to make and use the same, this enablement being provided in particular for the subject matter of the appended claims, which make up a part of the original description.

Throughout the application, the wording “comprising one” or “having one” means “comprising at least one” or “having at least one” unless specified to the contrary.

As used herein, the words “a” and “an” and the like carry the meaning of “one or more.”

The phrases “selected from the group consisting of,” “chosen from,” and the like include mixtures of the specified materials. Terms such as “contain(s)” and the like are open terms meaning ‘including at least’ unless otherwise specifically noted.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein. In this regard, certain embodiments within the invention may not show every benefit of the invention, considered broadly.

1. A composition comprising, in a continuous aqueous phase:
an aqueous dispersion of polyurethane, the dispersed polyurethane comprising the reaction products of:
A) a prepolymer according to the formula: 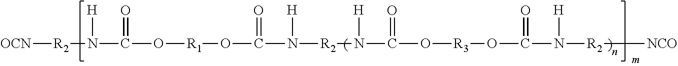
where
R₁ represents a hydrocarbon-based radical derived from a polyester polyol, and in particular from a polyester diol,
R₂ represents a hydrocarbon-based radical derived from an aliphatic or cycloaliphatic polyisocyanate,
R₃ represents a hydrocarbon-based radical derived from a diol, optionally of low molecular weight, optionally substituted by ionic groups,
n is equal to 0 to 5, and
m is >1;
B) at least one chain extender according to the formula:
H₂N—R₄—NH₂
where
R₄ represents an alkylene or alkylene oxide radical that is not substituted by ionic or potentially ionic groups; and
C) at least one chain extender according to the formula:
H₂N—R₅—NH₂
where
R₅ represents an alkylene radical substituted by ionic or potentially ionic groups;
said polyurethane being present in an amount of solids greater than or equal to 5% by weight relative to the total weight of said composition,
said composition comprising an emulsifying system comprising less than 1% by weight of triethanolamine and comprising a dyestuff chosen from pulverulent materials.

2. The composition according to claim 1, in which said polyurethane is present in an amount of solids inclusively between 5% and 15% by weight relative to the total weight of said composition.

3. The composition according to claim 1, in which said polyurethane comprises compounds situated at the ends of the chains and terminating said chains, that are derived from compounds having the formula: 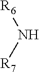
in which R₆ represents a hydrogen atom or an alkylene radical optionally having a hydroxyl end and R₇ represents an alkylene radical optionally having a hydroxyl end.

4. The composition according to claim 1, in which the radical R₁ is obtained by (poly)condensation of at least one dicarboxylic acid with at least one diol.

5. The composition according to claim 1, in which the radical R₂ is derived from a cycloaliphatic polyisocyanate.

6. The composition according to claim 1, in which the radical R₃ is derived from neopentyl glycol.

7. The composition according to claim 1, in which the radical R₄ is chosen from ethylenediamine, diethanolamine and mixtures thereof.

8. The composition according to claim 1, in which the radical R₅ is chosen from diaminosulphonates.

9. The composition according to claim 1, in which said composition comprises an emulsifying system chosen from:
i) an alkali metal alkylphosphate or phosphine oxide of the formula (R—O)ₙ—P═O(O.M)ₘ with R representing a linear or branched C₈-C₂₂ alkyl group, n being equal to 1, 2 or 3 and m being equal to 0, 1 or 2, with m+n being equal to 3 and M representing a hydrogen atom or an alkali or alkaline-earth metal;
ii) a polyethoxylated alcohol of formula R′—(OCH₂CH₂)ₚ—OH with R′ representing a linear or branched C₁-C₃₀ alkyl and p representing an integer inclusively between 2 and 30;
iii) a glutamic acid salt of formula R—CONH—C(COO⁻M)-C₂H₄—COO-M′ with R representing a linear or branched C₈-C₂₂ alkyl group and M′ representing an alkali or alkaline-earth metal; and
iv) an alkyl glucoside obtained by condensation of glucose and of linear or branched C₈-C₂₂ fatty alcohols.

10. The composition according to claim 1, in which the emulsifying system comprises at least one surfactant chosen from potassium cetyl phosphate, steareth-2, steareth-20 and mixtures thereof.

11. The composition according to claim 1, in which the emulsifying system comprises at least one surfactant chosen from sodium stearoyl glutamate and cetylstearyl glucoside, and mixtures thereof.

12. The composition according to claim 1, additionally comprising at least one hydrophilic thickener chosen from the copolymers derived from the polymerization:
(i) of at least one monomer of formula (1) below: 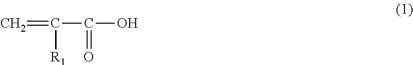
in which, R₁ denotes H or CH₃ or C₂H₅, that is to say acrylic acid, methacrylic acid or ethacrylic acid monomers, and
(ii) of at least one monomer of (C₁₀-C₃₀)alkyl ester of unsaturated carboxylic acid type corresponding to the monomer of formula (2) below: 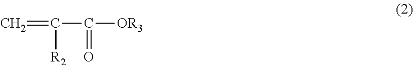
in which, R₂ denotes H or CH₃ or C₂H₅ and preferably H or CH₃, R₃ denoting a C₁₀-C₃₀ alkyl radical.

13. The composition according to claim 12, in which said hydrophilic thickener is chosen from polymers resulting from the polymerization of a mixture of monomers comprising:
(i) essentially acrylic acid,
(ii) an ester of formula (2) described above in which R₂ denotes H or CH₃, R₃ denoting an alkyl radical having from 12 to 22 carbon atoms, and
(iii) a crosslinking agent.

14. The composition according to claim 1, further comprising at least one lipophilic plasticizer present in an amount by weight greater than or equal to 2% relative to the total weight of the composition.

15. The composition according to claim 14, in which said plasticizer is chosen from:
the (poly)esters derived from the reaction(s) of at least one carboxylic acid with at least one (poly)ol,
glycol ethers,
N-ethyl-o,p-toluenesulphonamide,
carbonates,
ketones,
and mixtures thereof.
